(19) United States
(12) Patent Application Publication
(10) Pub. No.: US 2004/0236110 A1
(43) Pub. Date: Nov. 25, 2004

(54) Substituted 3-pyridyl indoles and indazoles as c17,20 lyase inhibitors

(76) Inventors: Gaetan H Ladouceur, Guilford, CT (US); Michael Burke, New Haven, CT (US); Wai C Wong, Trumbull, CT (US); Donald E Bierer, Bethany, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/491,214

(22) PCT Filed: Sep. 26, 2002
(86) PCT No.: PCT/US02/30482

Related U.S. Application Data
(60) Provisional application No. 60/324,993, filed on Sep. 26, 2001.

(51) Int. Cl. C 07D 4 3/02; C07D 209/12

(52) U.S. Cl. 546/277.4; 548/502

(57) ABSTRACT

The invention provides novel substituted 3-pyridyl indoles and indazoles and pharmaceutical compositions thereof. The invention also provides methods of use of substituted 3-pyridyl indoles and indazoles and pharmaceutical compositions thereof as inhibitors of lyases, e.g., the 17α-hydroxylase-C17,20-lyase enzyme. The invention further provides methods for the treatment of cancer in a subject, comprising administering a substituted 3-pyridyl indoles and indazoles or a pharmaceutical composition comprising a substituted 3-pyridyl indoles and indazoles to a subject. The cancer can be, e.g., prostate cancer or breast cancer.

### BACKGROUND OF THE INVENTION

Steroid biosynthesis begins in cells of the adrenal gland where the initial product in sterol biosynthesis, cholesterol, is converted into the adrenal steroid hormones aldosterone, hydrocortisone, and corticosterone by a series of P₄₅₀-mediated hydroxylation steps. The cholesterol side-chain cleavage activity that represents the first step in steroid hormone biosynthesis is a P₄₅₀-mediated oxidation and cleavage of a pair of adjacent methylene groups to two carbonyl fragments, pregnenolone and isocaprylaldehyde (see Walsh, Enzymatic Reaction Mechanisms, W.H. Freeman and Company: pp. 474-77, 1979). Another critical set of enzymatic conversions in steroid metabolism is facilitated by 17α-hydroxylase-17,20-lyase (CYP17, P₄₅₀ 17). CYP17 is a bifunctional enzyme which possesses both a C17,20-lyase activity and a C17-hydroxylase activity. Significantly, these two alternative enzymatic activities of CYP17 result in the formation of critically different intermediates in steroid biosynthesis and each activity appear to be differentially and developmentally regulated (see e.g. l'Allemand et al. Eur. J. Clin. Invest. 2000, 30, 28-33).

The C17,20-lyase activity of CYP17 catalyzes the conversion of 17α-hydroxy-pregnenolone and 17α-hydroxy-progesterone to dehydroepiandrosterone (DHEA) and delta-4-androstenedione (androstenedione) respectively. Both DHEA and androstenedione lyase products are key intermediates in the synthesis of not only the androgens testosterone and dihydrotestosterone (DHT), but also the estrogens 17β-estradiol and estrone. Indeed, adrenal and ovarian estrogens are the main sources of estrogens in postmenopausal women (see e.g. Harris et al. Br. J. Cancer 1988, 58, 493-6). In contrast, the C17-hydroxylase activity of CYP17 catalyzes the conversion of the common intermediate progesterone to 17-hydroxyprogesterone, a precursor of cortisol. Therefore the first activity of CYP17, the C17-hydroxylase activity, promotes the formation of glucocorticoids while the second activity of CYP17, the C17,20-lyase activity, promotes the formation of sex hormones—particularly androgens including testosterone as well as estrogens.

Prostate cancer is currently one of the most frequently diagnosed forms of cancer in men in the U.S. and Europe. Prostate cancer is typically androgen-dependent and, accordingly, the reduction in androgen production via surgical or pharmacological castration remains the major treatment option for this indication. However, complete rather than partial withdrawal of androgens may be more effective in treating prostate cancer (Labrie, F. et al., Prostate 1983, 4, 579 and Crawford, E. D. et al., N. Engl. J. Med. 1989, 321, 419). Pharmacological inhibition of CYP17 may be a promising alternative treatment to antiandrogens and LHRH agonists in that testicular, adrenal, and peripheral androgen biosynthesis would be reduced rather than only testicular androgen production (Njar, V. et al., J. Med. Chem. 1998, 41, 902). One such CYP17 inhibitor, the fungicide ketoconazole, has been used previously for prostate cancer treatment (Trachtenberg, J., J. Urol. 1984, 132, 61 and Williams, G. et al, Br. J. Urol. 1986, 58, 45). However, this drug is a relatively non-selective inhibitor of cytochrome P450 (CYP) enzymes, has weak CYP17 activity, and has a number of notable side effects associated with it including liver damage (De Coster, R. et al., J Steroid Biochem. Mol. Biol. 1996, 56, 133 and Lake-Bakaar, G. et al., Br. J. Med. 1987, 294, 419).

The importance of potent and selective inhibitors of CYP17 as potential prostate cancer treatments has been the subject of numerous studies and reviews (Njar, V. et al., Curr. Pharm. Design, 1999, 5, 163; Barrie, S. E. et al, Endocr. Relat. Cancer 1996, 3, 25 and Jarman, M. et al., Nat. Prod. Rep. 1998, 495). Finasteride, a 5α-reductase inhibitor, is an approved treatment for benign prostatic hyperplasia (BPH), although it is only effective with patients exhibiting minimal disease. While finasteride reduces serum DHT levels, it increases testosterone levels, and may therefore be insufficient for prostate cancer treatment (Peters, D. H. et al., Drugs, 1993, 46, 177). Certain anti-androgenic steroids, for example, cyproterone acetate (17α-acetoxy-6-chloro-1α,2α-methylene-4,6-pregnadiene-3,20-dione), have been tested as adjuvant treatments for prostate cancer. Many other steroids have been tested as hydroxylase/lyase inhibitors. See, for example, PCT Specification WO 92/00992 (Schering AG) which describes anti-androgenic steroids having a pyrazole or triazole ring fused to the A ring at the 2,3-position, or European specifications EP-A288053 and EP-A413270 (Merrell Dow) which propose 170-cyclopropylamino-androst-5-en-313-ol or -4-en-3-one and their derivatives.

In addition to the use of CYP17 inhibitors in the treatment of prostate cancer, a second potential indication would be for estrogen-dependent breast cancer. In postmenopausal patients with advanced breast cancer, treatment with high doses of ketoconazole resulted in suppression of both testosterone and estradiol levels, implicating CYP17 as a potential target for hormone therapy (Harris, A. L. et al., Br. J. Cancer 1988, 58, 493).

Chemotherapy is usually not highly effective, and is not a practical option for most patients with prostate cancer because of the adverse side effects which are particularly detrimental in older patients. However, the majority of patients initially respond to hormone ablative therapy although they eventually relapse, as is typical with all cancer treatments (McGuire, in: Hormones and Cancer, Iacobelli et al Eds.; Raven Press: New York, 1980, vol. 15, pp. 337-344). Current treatment by orchidectomy or administration of gonadotropin-releasing hormone (GnRff) agonists results in reduced androgen production by the testis, but does not interfere with androgen synthesis by the adrenals. Following three months of treatment with a GnRH agonist, testosterone and DHT concentrations in the prostate remained at 25% and 10%, respectively, of pretreatment levels (Forti et al., J. Clin. Endocrinol. Metab. 1989, 68, 461). Similarly, about 20% of castrated patients in relapse had significant levels of DHT in their prostatic tissue (Geller et al., J. Urol. 1984, 132, 693). These findings suggest that the adrenals contribute precursor androgens to the prostate. This is supported by clinical studies of patients receiving combined treatment with either GNRH or orchidectomy and an anti-androgen, such as flutamide, to block the actions of androgens, including adrenal androgens. Such patients have increased progression-free survival time compared to patients treated with GnRH agonist or orchidectomy alone (Crawford et al., N. Engl. J. Med. 1989, 321, 419 and Labrie et al., Cancer Suppl. 1993, 71, 1059).

Although patients initially respond to endocrine therapy, they frequently relapse. It was reported recently that in 30% of recurring tumors of patients treated with endocrine therapy, high-level androgen receptor (AR) amplification was found (Visakorpi, et al., Nature Genetics 1995, 9, 401). Also, flutamide tends to interact with mutant ARs, and stimulate prostatic cell growth. This suggests that AR amplification may facilitate tumor cell growth in low androgen concentrations. Thus, total androgen blockade as first line therapy may be more effective than conventional androgen deprivation by achieving maximum suppression of androgen concentrations which may also prevent AR amplification. It is presently unclear whether sequential treatment with different agents can prolong the benefits of the initial therapy. This strategy has been found effective in breast cancer treatment. New agents which act by different mechanisms could produce second responses in a portion of relapsed patients. Although the percentage of patients who respond to second-line hormonal therapy may be relatively low, a substantial number of patients may benefit because of the high incidence of prostate cancer. Furthermore, there is the potential for developing more potent agents than current therapies, none of which are completely effective in blocking androgen effects.

The need exists for C17,20-lyase inhibitors that overcome the above-mentioned deficiencies.

### SUMMARY OF THE INVENTION

The invention provides substituted 3-pyridyl indole and indazole compounds which inhibit the lyase activity of enzymes, e.g., 17α-hydroxylase-C17,20-lyase. Indole compounds of the invention have the formula

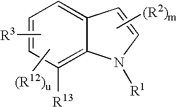

in which

R¹ represents

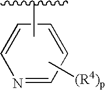

in which R⁴ represents C₁₋₄ alkyl; and p is 0, 1, or 2;

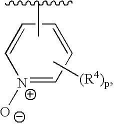

provided that R³ is other than a pyridyl or an N-oxide-containing group; or

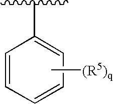

in which R¹ represents CN, halogen, CHO, or C(O)N(6)₂ in which R¹ represents H or C₁₋₄ alkyl; and q is 0, 1, or 2.

R² represents C₁₋₄ alkyl; and m is 0, 1, or 2.

R³ represents

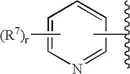

in which R⁷ is C₁₋₄ alkyl or CN; and r is 0, 1, or 2;

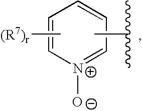

provided that R¹ is other than a pyridyl or an N-oxide-containing group;

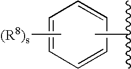

in which

R⁸ represents

CN,

halogen,

C₁₋₄ alkyl,

C₁₋₄ alkoxy,

NO₂,

CF₃,

C₁₋₄ acyl,

CO₂R⁹ wherein R⁹ is H or C₁₋₄ alkyl, or

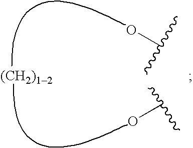

and

s is 0, 1, or 2;

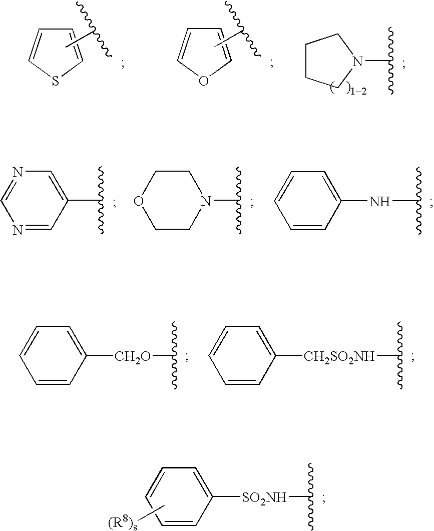

C₁₋₄ alkyl-SO₂NH—;

CN;

N(R¹⁰)₂, wherein R¹⁰ is C₁₋₄ alkyl; or

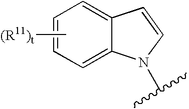

in which R¹¹ is halogen; and t is 0, 1, or 2.

R¹² represents C₁₋₄ alkyl, C₁₋₄ alkoxy, halogen, or CN provided that R³ is other than cyano; and u is 0, 1, or 2.

R¹³ represents H or R¹¹².

Furthermore, one of R¹ and R³ is a 3-pyridyl or 3-pyridyl-N-oxide group which is unsubstituted at the 2- and 6-positions. Pharmaceutically acceptable salts of these compounds are also within the scope of the invention. Indazole compounds of the invention have the formula

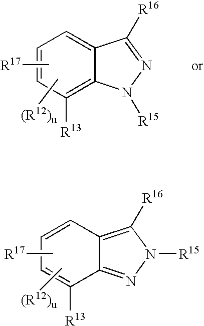

in which

R¹² represents C₁₋₄ alkyl, C₁₋₄ alkoxy, halogen, or CN; and u is 0, 1, or 2.

R¹³ represents H or R¹².

R¹⁵ represents

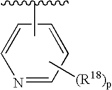

in which R¹⁸ represents C₁₋₄ alkyl; and p is 0, 1, or 2; or

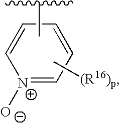

provided that R¹⁷ is other than a pyridyl or an N-oxide-containing group.

R¹⁶ represents H or C014 alayl.

R¹⁷ represents

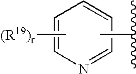

in which R¹⁹ is C₁₋₄ alkyl; and r is 0, 1, or 2; or

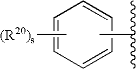

in which R²⁰ represents halogen; C₁₋₄ alkyl, C₁₋₄ alkoxy, NO₂, CF₃, or CO₂R²¹ in which R²¹ is H or Ca₄ alkyl; and s is 0, 1, or 2.

Furthermore, one of R¹⁵ and R¹⁷ is a 3-pyridyl or 3-pyridyl-N-oxide group which is unsubstituted at the 2- and 6-positions. Pharmaceutically acceptable salts of these materials are also within the scope of the invention.

The invention also provides pharmaceutical compositions for inhibiting lyase activity, comprising a compound of the invention and a pharmaceutically acceptable carrier.

The invention also provides methods for inhibiting lyases, comprising contacting the lyase with a compound of the invention. More particularly, the invention provides a method of inhibiting a 17α-hydroxylase-C17,20 lyase, comprising contacting a 17α-hydroxylase-C17,20 lyase with a compound of the invention.

The invention further provides methods for treating diseases which can benefit from an inhibition of a lyase enzyme. Exemplary diseases are lyase-associated diseases, e.g., diseases resulting from an excess of androgens or estrogens. For example, the invention provides a method for treating cancer in a subject, comprising administering to the subject a pharmaceutically effective amount of a compound of the invention, such that the cancer is treated.

The method of treatment may be applied where the subject is equine, canine, feline, or a primate, in particular, a human.

The cancer may, for example, be prostate or breast cancer. Accordingly, a method for treating prostate cancer in a subject, comprises administering to the subject a therapeutically effective amount of a compound of the invention, such that the prostate cancer in the subject is treated. Similarly, a method for treating breast cancer in a subject comprises administering to the subject a therapeutically effective amount of a compound of the invention, such that the breast cancer in the subject is treated.

### DETAILED DESCRIPTION OF TIRE INVENTION

The invention is based at least in part on the discovery that substituted 3-pyridyl indole and indazole compounds inhibit the enzyme 17α-hydroxylase-C17,20-lyase.

In a preferred embodiment, indole compounds of the invention have the formula

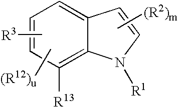

in which

R¹ represents

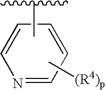

in which R⁴ represents C₁₋₄ alkyl; and p is 0, 1, or 2;

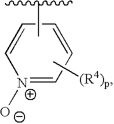

provided that R³ is other than a pyridyl or an N-oxide-containing group; or

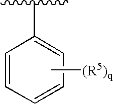

in which R⁵ represents CN, halogen, CHO, or C(O)N(R⁶)₂ in which R⁶ represents H or C₁₋₄ alkyl; and q is 0, 1, or 2.

R² represents C₁₋₄ alkyl; and m is 0, 1, or 2.

R³ represents

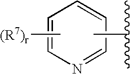

in which R⁷ is C₁₋₄ alkyl or CN; and r is 0, 1, or 2;

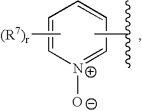

provided that R¹ is other than a pyridyl or an N-oxide-containing group;

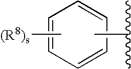

in which

R⁸ represents

CN,

halogen,

C₁₋₄ alkyl,

C₁₋₄ alkoxy,

NO₂,

CF₃,

C₁₋₄ acyl,

CO₂R⁹ wherein R⁹ is H or C₁₋₄ alkyl, or

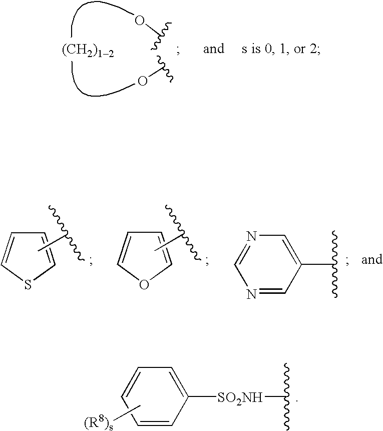

R¹² represents C₁₋₄ alkyl, C₁₋₄ alkoxy, halogen, or CN provided that R³ is other than cyano; and u is 0, 1, or 2.

R¹³ represents H or R¹².

Furthermore, one of R¹ and R³ is a 3-pyridyl or 3-pyridyl-N-oxide group which is unsubstituted at the 2- and 6-positions. Pharmaceutically acceptable salts of these compounds are also within the scope of the invention.

In a more preferred embodiment, indole compounds of the invention have the formula

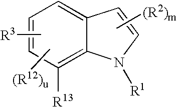

in which

R¹ represents

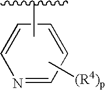

in which R⁴ represents C₁₋₄ alkyl; and p is 0, 1, or 2;

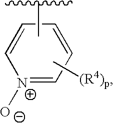

provided that R³ is other than a pyridyl or an N-oxide-containing group; or

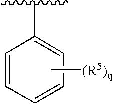

in which R⁵ represents CN, halogen, CHO, or C(O)N(R⁶)₂ in which R⁶ represents H or C₁₋₄ alkyl; and q is 0, 1, or 2.

R² represents C₁₋₄ alkyl; and m is 0, 1, or 2.

R³ represents

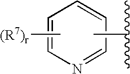

in which R⁷ is C₁₋₄alkyl or CN; and r is 0, 1, or 2;

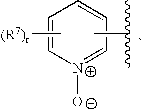

provided that R¹ is other than a pyridyl or an N-oxide-containing group;

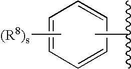

in which

R⁸ represents

CN,

halogen,

C₁₋₄ alkyl,

C₁₋₄ alkoxy,

NO₂,

CF₃,

C₁₋₄ acyl,

CO₂R⁹ wherein R⁹ is H or C₁₋₄ alkyl, or

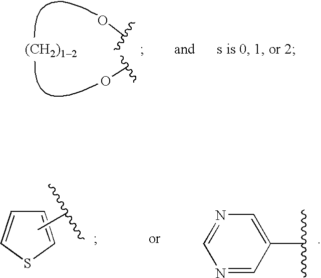

R¹² represents C₁₋₄ alkyl, C₁₋₄ alkoxy, halogen, or CN provided that R³ is other than cyano; and u is 0, 1, or 2.

R¹³ represents H or R¹².

Furthermore, one of R¹ and R³ is a 3-pyridyl or 3-pyridyl-N-oxide group which is unsubstituted at the 2- and 6-positions. Pharmaceutically acceptable salts of these compounds are also within the scope of the invention.

In a most preferred embodiment, indole compounds of the invention have the formula,

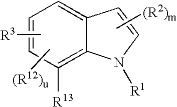

in which

R¹ represents

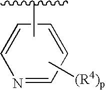

in which R⁴ represents C₁₋₄ alkyl; and p is 0, 1, or 2;

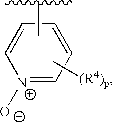

provided that R³ is other than a pyridyl or an N-oxide-containing group; or

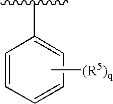

in which R⁵ represents CN, halogen, CHO, or C(O)N(R⁶)₂ in which R⁶ represents H or C₁₋₄ alkyl; and q is 0, 1, or 2.

R² represents C₁₋₄ alkyl; and m is 0, 1, or 2.

R³ represents

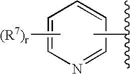

in which R⁷ is C₁₋₄ alkyl or CN; and r is 0, 1, or 2;

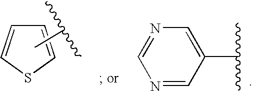

R¹² represents C₁₋₄ alkyl, C₁₋₄ alkoxy, halogen, or CN provided that R³ is other than cyano; and u is 0, 1, or 2.

R¹³ represents H or R¹².

Furthermore, R¹ is a 3-pyridyl or 3-pyridyl-N-oxide group which is unsubstituted at the 2- and 6-positions. Pharmaceutically acceptable salts of these compounds are also within the scope of the invention.

In a preferred embodiment, indazole compounds of the invention have the formula

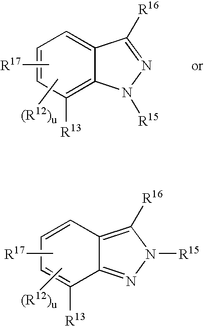

in which

R¹² represents C₁₋₄ alkyl, C₁₋₄ alkoxy, halogen, or CN; and u is 0, 1, or 2.

R¹³ represents H or R¹².

R¹⁵ represents

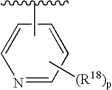

in which R¹⁸ represents Clot alkyl; and p is 0, 1, or 2; or

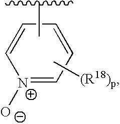

provided that R¹⁷ is other than a pyridyl or an N-oxide-containing group.

R¹⁶ represents H.

R¹⁷ represents

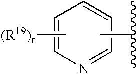

in which R¹⁹ is C₁₋₄ alkyl; and r is 0, 1, or 2; or

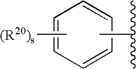

in which R²⁰ represents halogen; C₁₋₄ alkyl, C₁₋₄ alkoxy, NO₂, CF₃, or CO₂R²¹¹ in which R²¹ is H or C₁₋₄ alkyl; and s is 0, 1, or 2.

Furthermore, one of R¹⁵ and R¹⁷ is a 3-pyridyl or 3-pyridyl-N-oxide group which is unsubstituted at the 2- and 6-positions. Pharmaceutically acceptable salts of these materials are also within the scope of the invention.

In a more preferred embodiment, indazole compounds of the invention have the formula

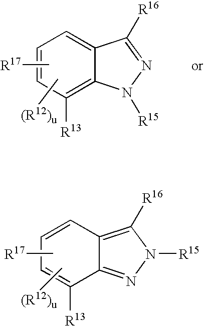

in which

R¹² represents C₁₋₄ alkyl, CIA alkoxy, halogen, or CN; and u is 0, 1, or 2.

R¹³ represents H or R¹².

R¹⁵ represents

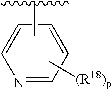

in which R¹⁸ represents C₁₋₄ alkyl; and p is 0, 1, or 2;

R¹⁶ represents H.

R¹⁷ represents

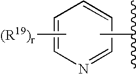

in which R¹⁹ is C₁₋₄ alkyl; and r is 0, 1, or 2;

Furthermore, one of R¹⁵ and R¹⁷ is a 3-pyridyl group which is unsubstituted at the 2- and & positions. Pharmaceutically acceptable salts of these materials are also within the scope of the invention.

Definitions

For convenience, certain terms employed in the specification, examples, and appended claims are collected here.

The term “agonist” of an enzyme refers to a compound that binds to the enzyme and stimulates the action of the naturally occurring enzyme, or a compound which mimics the activity of the naturally occurring enzyme.

The term “antagonist” of an enzyme refers to a compound that binds to the enzyme and inhibits the action of the naturally occurring enzyme.

The term “analog” of a compound refers to a compound having a some structural similarity to a particular compound and having essentially the same type of biological activity as the compound.

The term “CYP17 substrate” includes any of the various steroid hormones acted upon by a CYP17 or a CYP17-like P₄₅₀ enzyme. Examples include pregnenolone, progesterone and their 17α-hydroxylated forms. Pregnenolone is converted to DHEA via a CYP17 C17,20-lyase reaction, but is also subject to C17α-hydroxylation via the C17,20-lyase activity. Progesterone is converted to delta 4-androstenedione via a CYP17 C17,20-lyase reaction, but is also subject to C17α-hydroxylation via the C17-hydroxylase activity to form 17-hydroxy-progesterone, a precursor to hydrocortisone (i.e. cortisol).

The term “CYP17 metabolite” refers to any of the steroid hormones that are synthesized from a cholesterol precursor via a CYP17-mediated reaction, such as a C17-hydroxylase reaction or a C17,20-lyase reaction. Examples of CYP17 metabolites include the androgens, such as testosterone, which are synthesized via a CYP17 C17,20-lyase reaction from CYP17 substrate precursors such as pregnenolone (converted to DHEA by the CYP17 C17,20-lyase activity), and progesterone (converted to delta 4-androstenedione by the CYP17 C17,20-lyase activity). Progestagens such as progesterone are primarily synthesized in the corpus luteum. The androgens are responsible for, among other things, development of male secondary sex characteristics and are primarily synthesized in the testis. Other examples include the estrogens, which are also synthesized from a cholesterol precursor via a CYP17-mediated reaction. The estrogens are responsible for, among other things, the development of female secondary sex characteristics and they also participate in the ovarian cycle and are primarily synthesized in the ovary. Another group of CYP17 metabolites are the glucocorticoids, such as hydrocortisone (i.e. cortisol), which is synthesized from progesterone via a CYP17-mediated reaction. The glucocorticoids, among other functions, promote gluconeogenesis and the formation of glycogen and also enhance the degradation of fat. The glucocorticoids are primarily synthesized in the adrenal cortex.

The term “CYP17 metabolite” is further meant to include other steroid hormones which, although not necessarily synthesized by a CYP17-mediated reaction, may nonetheless be understood by the skilled artisan to be readily affected by an alteration in a CYP17-mediated activity. For example, the mineralocorticoids, such as aldosterone, are derived from cholesterol via a progesterone intermediate. Since progesterone is also converted to the glucocorticoids and sex steroids via CYP17-mediated reactions, an alteration of a CYP17 activity can alter the amount of progesterone available for conversion to aldosterone. For example, inhibition of CYP17 activity can increase the amount of progesterone available for conversion into aldosterone. Therefore, inhibition of CYP17 can lead to an increase in the level of aldosterone. The mineralocorticoids function, among other things, to increase reabsorption of sodium ions, chloride ions, and bicarbonate ions by the kidney, which leads to an increase in blood volume and blood pressure. The mineralocorticoids are primarily synthesized in the adrenal cortex.

The term “CYP17 metabolite-associated disease or disorder” refers to a disease or disorder which may be treated by alteration of the level of one or more CYP17 metabolites. Examples include a hormone dependent cancer, such as an androgen-dependent prostate cancer, which may be treated by inhibiting CYP17-mediated androgen synthesis, and an estrogen-dependent breast cancer or ovarian cancer, which may be treated by inhibiting CYP17-mediated estrogen synthesis. Other examples of “CYP17 metabolite-associated diseases or disorders” are Cushing's disease, hypertension, prostatic hyperplasia, and glucocorticoid deficiency. Patients with Cushing's syndrome are relatively insensitive to glucocorticoid feedback and exhibit an oversecretion of cortisol devoid of a circadian cycle (see e.g. Newell-Price & Grossman, Ann. Endocrinol. 2001, 62, 173-9). Another CYP17 metabolite-associated disease or disorder is hypertension Mineralocorticoid excess causes hypertension by facilitating the sodium retention at renal tubules.

The term “derivative” of a compound refers to another compound which can be derived, e.g., by chemical synthesis, from the original compound. Thus a derivative of a compound has certain structural similarities with the original compound.

“Disease associated with an abnormal activity or level of a lyase” refers to diseases in which an abnormal activity or protein level of a lyase is present in certain cells, and in which the abnormal activity or protein level of the lyase is at least partly responsible for the disease.

A “disease associated with a lyase” refers to a disease that can be treated with a lyase inhibitor, such as the compounds disclosed herein.

A “lyase” refers to an enzyme having a lyase activity.

“Lyase activity” refers to the activity of an enzyme to catalyze the cleavage of the bond C17-C20 in 17α-hydroxy-pregnenolone and 17α-hydroxy-progesterone to form dehydroepiandrosterone (DHEA) and delta4-androstenedione, respectively. Lyase activity also refers to the cleavage of a similar bond in related compounds.

A “lyase inhibitor” is a compound which inhibits at least part of the activity of a lyase in a cell. The inhibition can be at least about 20%, preferably at least about 40%, even more preferably at least about 50%, 70%, 80%, 90%, 95%, and most preferably at least about 98% of the activity of the lyase.

A “patient” or “subject” to be treated by the subject method can mean either a human or non-human animal.

“Treating” a disease refers to preventing, curing or improving at least one symptom of a disease.

The following definitions pertain to the chemical structure of compounds:

The term “heteroatom” as used herein means an atom of nitrogen, oxygen, or sulfur.

The term “alkyl” refers to the radicals of saturated aliphatic groups, including straight-chain alkyl groups and branched-chain alkyl groups.

The term “cycloalkyl” (alicyclic) refers to radicals of cycloalkyl compounds, examples being cyclopropyl, cyclobutyl, cyclopentyl, cyclohexyl, etc.

Unless the number of carbons is otherwise specified, “lower alkyl” as used herein means an alkyl group but having from one to six carbons, preferably from one to four carbon atoms in its backbone structure. Preferred alkyl groups are lower alkyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “alkoxyl” or “alkoxy” as used herein refer to moiety in which an alkyl group is bonded to an oxygen atom, which is in turn bonded to the rest of the molecule. Examples are methoxy, ethoxy, propyloxy, tert-butoxy, etc.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; (i.e., J. Org. Chem. 2002, 67(1), 24A. The abbreviations contained in said list, and all abbreviations utilized by organic chemists of ordinary skill in the art are hereby incorporated by reference.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. Protective Groups in Organic Synthesis, 3^{rd} ed.; Wiley: New York, 1999).

### Abbreviations and Acronyms

When the following abbreviations are used throughout the disclosure, they have the following meaning:

| | |
|---|---|
| ACN | acetonitrile |
| AcOH | acetic acid |
| Ar | argon |
| BINAP | 2,2′-bis(diphenylphosphino)1,1′-binaphthyl |
| BSA | bovine serum albumin |
| n-BuLi | n-butyllithium |
| CDCl₃ | chloroform-d |
| CD₃OD | methanol-d₄ |
| CHCl₃ | chloroform |
| CH₂Cl₂ | methylene chloride |
| CH₃CN | acetonitrile |
| CuI | copper iodide |
| Cs₂CO₃ | cesium carbonate |
| CPM | counts per minute |
| DME | 1,2-dimethoxyethane |
| DMF | dimethylformamide |
| DMSO | dimethylsulfoxide |
| EPA | Environmental Protection Agency (as in EPA vial) |
| ES-MS | eletrospray mass spectrometry |
| Et₃N | triethylamine |
| EtOAc | ethyl acetate |
| Et₂O | diethyl ether |
| EtOH | ethanol |
| GCEI | gas chromatography - electron impact mass spectrometry |
| GCMS | gas chromatography/mass spectrometry |
| H₂ | hydrogen gas |
| HCl | hydrochloric acid |
| ¹H NMR | proton nuclear magnetic resonance |
| HEPES | 4-(2-Hydroxyethyl)piperazine-1-ethanesulfonic acid |
| Hex | Hexane |
| HPLC | high performance liquid chromatography |
| I.D. | internal diameter |
| KOH | potassium hydroxide |
| LCMS | liquid chromatography/mass spectroscopy |
| M + 1 | exact mass + 1 |
| MeCN | acetonitrile |
| MeOH | methanol |
| min. | minute |
| mmol | millimole |
| mg | milligram |
| mL | milliliter |
| NaOtBu | sodium tert-butoxide |
| Na₂CO₃ | sodium carbonate |
| NaH | sodium hydride |
| NaHCO₃ | sodium bicarbonate |
| NaHMDS | sodium bis(trimethylsilyl)amide |
| Na₂SO₄ | sodium sulfate |
| NH₃ | ammonia |
| NH₄Cl | ammonium chloride |
| NH₄OH | ammonium hydroxide |
| Pd/C | palladium on carbon |
| Pd₂(dba)₃ | tris(dibenzylideneacetone)dipalladium(0) |
| Pd(dppf)₂Cl₂ | [1,1′-bis(diphenylphosphino)ferrocene]dichloropalladium(II) |
| Pd(PPh₃)₄ | tetrakis(triphenylphosphine)palladium(0) |
| POCl₃ | Phosphorous oxychloride |
| Rf | TLC retention coefficient |
| SPA | Scintillation Proximity Assay |
| THF | tetrahydrofuran |
| TFA | trifluoroacetic acid |
| TMS | tetramethylsilane |
| TLC | thin layer chromatography |
| Rt | HPLC retention time |

Compounds of the Invention

The present invention is directed to compounds which inhibit 17α-hydroxylase-C17,20-lyase.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivatizaton with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Compounds may contain a basic functional group, such as amino or alkylamino, and are, thus, capable of forming pharmaceutically acceptable salts with pharmaceutically acceptable acids. The term “pharmaceutically acceptable salts” in this respect, refers to the relatively nontoxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fulmarate, succinate, tartrate, napthylate, mesylate, glucoheptonate, lactobionate, and laurylsulphonate salts and the like. (See, for example, Berge et al., “Pharmaceutical Salts”, J. Pharm. Sci. 1977, 66, 1-19).

Pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pahmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. These salts can be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Contemplated equivalents of the compounds described above include compounds which otherwise correspond thereto, and which have the same general properties thereof (e.g., functioning as 17,20-hydroxylase-C17,20-lyase inhibitors), wherein one or more simple variations of substituents are made which do not adversely affect the efficacy of the compound in binding to 17α-hydroxylase-C17,20-lyase receptors. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

Diseases that Can be Treated With the Compounds of the Invention

The present invention provides a method of inhibiting a lyase, e.g., 17α-hydroxylase-C17,20-lyase, comprising contacting a lyase with a compound of the invention. The activity can be inhibited by at least 20%, preferably at least about 50%, more preferably at least about 60%, 70%, 80%, 90%, 95%, and most preferably at least about 98%. In one embodiment, the invention provides a method for inhibiting a lyase in vitro. In a preferred embodiment, the lyase is in vivo or ex vivo. For example, the invention provides methods for inhibiting a lyase in a cell, comprising contacting the cell with a compound of the invention, such that the activity of the lyase is inhibited. The cell may further be contacted with a composition stimulating the uptake of the compound into the cell, e.g., liposomes. In one embodiment, the invention provides a method for inhibiting a lyase in a cell of a subject, comprising administering to the subject a therapeutically effective amount of a compound of the present invention, or a formulation comprising a compound of the present invention, such that the lyase is inhibited in a cell of the subject. The subject can be one having a disease associated with a lyase, e.g., cancer. Preferred types of cancer that can be treated according to the invention include prostate cancer and breast cancer. Other diseases that can be treated include diseases in which it is desired to prevent or inhibit the formation of a hormone selected from the group consisting of the androgens testosterone and dihydrotestosterone (DHT) and the estrogens 17-estradiol and estrone. Generally, any disease that can be treated by inhibiting the activity of a lyase, e.g., 17α-hydroxylase-C17,20-lyase, can be treated with the compounds of the invention.

In general, the invention provides methods and compositions for the treatment of CYP17 metabolite-associated diseases and disorders. Examples include particularly sex steroid hormone dependent cancers, such as androgen-dependent prostate cancer, which may be treated by inhibiting CYP17-mediated androgen synthesis, and estrogen-dependent breast cancer or ovarian cancer, which may be treated by inhibiting CYP17-mediated estrogen synthesis.

For example, adenocarcinoma of the prostate is a common disease that causes significant morbidity and mortality in the adult male population (see Han and Nelson, Expert Opin. Pharmacother. 2000, 1, 443-9). Hormonal therapy for prostate cancer is considered when a patient fails with initial curative therapy, such as radical prostatectomy or definitive radiation therapy, or if he is found with an advanced disease. Hormonal agents have been developed to exploit the fact that prostate cancer growth is dependent on androgen. Non-steroidal anti-androgens (NSAAs) block androgen at the cellular level. Castration is another, albeit drastic means of decreasing androgens levels in order to treat or prevent prostate cancer. The methods and compositions of the invention are useful in inhibiting the C17,20-lyase activity of CYP17 and thereby decreasing levels of androgen production and the associated growth of androgen-dependent cancers such as prostate cancer.

In another example, breast cancer, particularly breast cancer in postmenopausal women, can be treated by administration of a C17,20-lyase inhibitor of the invention because adrenal and ovarian androgens are the main precursors of the estrogens which stimulate the growth of hormone dependent breast cancer. In addition, breast cancer can be treated with inhibitors of aromatase that prevent interconversion of estrogens and adrenal and ovarian androgens (see Harris et al., Eur. J. Cancer Clin. OticoL 1983, 19, 11). Patients failing to respond to aromatase inhibitors show elevated levels of androgens in response to aromatase inhibitor treatment (see Harris et al., Bi. J. Cancer 1988, 58, 493-6). Accordingly sequential blockade to inhibit androgen production as well as inhibit aromatase may produce greater estrogen suppression and enhanced therapeutic effects in treating breast and other estrogen hormone-dependent forms of cancer. Therefore the inhibitors of the invention may be used alone or in combination with other drugs to treat or prevent hormone-dependent cancers such as breast and prostate cancer.

Furthermore, susceptibility to prostate cancer and breast cancer has been associated with particular polymorphic alleles of the CYP17 gene (see e.g. McKean-Cowdin, Cancer Res. 2001, 61, 848-9; Haiman et al., Cancer Epidmeiol. Biomarkers 2001,10,743-8; Huang et al., Cancer Res. 2001, 59, 4870-5). Accordingly, the compositions of the invention are particularly suited to treating or preventing hormone-dependent cancers in individuals genetically predisposed to such cancers, particularly those predisposed due to an alteration in the CYP17 gene.

Another group of CYP17 metabolite-associated diseases or disorders amenable to treatment with the compositions and methods of the invention include those associated with mineralocorticoid excess such as hypertension caused by sodium retention at renal tubules. Such a mechanism operates in hypertension such as primary hyperaldosteronism and some forms of congenital adrenal hyperplasia. Recently, deficient cortisol metabolism in the aldosterone target organ has been recognized as a novel form of hypertension known as apparent mineralocorticoid excess. Disorders associated with mineralocorticoid synthesis include abnormalities of mineralocorticoid synthesis and/or metabolism which profoundly affect the regulation of electrolyte and water balance and of blood pressure (see e.g. Connell et al., Bailliere 's Best Pract. Res. Clin. Endocrinol. Metab. 2001, 15, 43-60). Characteristic changes in extracellular potassium, sodium and hydrogen ion concentrations are usually diagnostic of such disorders. Serious deficiency may be acquired, for example, in Addison's disease, or inherited. In most of the inherited syndromes, the precise molecular changes in specific steroidogenic enzymes have been identified. Mineralocorticoid excess may be caused by aldosterone or 11-deoxycorticosterone by inadequate conversion of cortisol to cortisone by 11-hydroxysteroid dehydrogenase type 2 in target tissues, by glucocorticoid receptor deficiency or by constitutive activation of renal sodium channels. Changes in electrolyte balance and renin as well as the abnormal pattern of corticosteroid metabolism are usually diagnostic. Where these abnormalities are inherited (e.g. 110- or 17α-hydroxylase deficiencies, glucocorticoid remediable hyperaldosteronism (GRA), receptor defects, Liddle's syndrome), the molecular basis is again usually known and, in some cases, may provide the simplest diagnostic tests. Primary aldosteronism, although readily identifiable, presents problems of differential diagnosis, important because optimal treatment is different for each variant. Finally, a significant proportion of patients with essential hypertension show characteristics of mild mineralocorticoid excess, for example low renin levels. As described above, a decrease in CYP17 activity can result in an alteration in mineralorticoid (e.g. aldosterone) biosynthesis. Accordingly, the “CYP17 metabolite-associated diseases or disorders” of the invention would include those associated with altered levels of aldosterone production (e.g. hypertension, primary adrenal hyperplasia).

Still other examples of CYP17 metabolite-associated diseases or disorders” are Cushing's disease, prostatic hyperplasia, glucocorticoid deficiency, and endometrial cancer.

The subject that can be treated according to the invention can be a mammal, e.g., a primate, equine, canine, bovine, ovine, porcine, or feline. In preferred embodiments of this method, the mammal is a human. In other embodiments, the invention provides methods for inhibiting the lyase activity of enzymes that are present in organisms other than mammals, e.g., yeast and fungus, e.g., mildew. Certain compounds of the invention may function as antifungal compounds.

Methods of Administering the Compounds of the Invention

The therapeutic methods of the invention generally comprise administering to a subject in need thereof, a pharmaceutically effective amount of a compound of the invention, or a salt, prodrug or composition thereof. The compounds of the invention can be administered in an amount effective to inhibit the activity of a 17α-hydroxylase-C17,20-lyase. The compounds of this invention may be administered to mammals, preferably humans, either alone or, preferably, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

Toxicity and therapeutic efficacy of the compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such reagents to the site of affected tissue in order to minimize potential damage to normal cells and, thereby, reduce side effects.

Data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of activity) as determined in cell culture. Such information can be used to more accurately determine useful doses in humans. The compounds of the invention have an IC₅₀ less than 10 μM as determined by the biochemical or cellular assay described herein. Some compounds of the invention are effective at concentrations of 10 nM, 100 nM, or 1 μM. Based on these numbers, it is possible to derive an appropriate dosage for administration to subjects.

Formation of prodrugs is well known in the art in order to enhance the properties of the parent compound Such properties include solubility, absorption, biostability and release time (see “Pharmaceutical Dosage Form and Drug Delivery Systems” 6th ed., Ansel et al., Ed.; Williams & Wilkins: pp. 27-29, 1995). Commonly used prodrugs of the disclosed compounds can be designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics 9th ed., Molinoffetal., Ed.; McGraw-Hill: pp. 11-13, 1996).

The pharmaceutical compositions can be prepared so that they may be administered orally, dermally, parenterally, nasally, ophthalmically, otically, sublingually, rectally or vaginally. Dermal administration includes topical application or transdermal administration. Parenteral adrministration includes intravenous, intraarticular, intramuscular, intraperitoneal, and subcutaneous injections, as well as use of infusion techniques. One or more compounds of the invention may be present in association with one or more non-toxic pharmaceutically acceptable ingredients and optionally, other active anti-proliferative agents, to form the pharmaceutical composition. These compositions can be prepared by applying known techniques in the art such as those taught in Remiigton's Pharmaceutical Sciences 14th ed., John E. Hoover, Managing Editor; Mack Publishing Co.: 1970 or Pharmaceutical Dosage Form and Drug Delivery Systems 6^{th} ed., Ansel et al., Ed.; Williams & Wilkins: 1995.

As indicated above, pharmaceutical compositions containing a compound of the invention may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically acceptable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia; and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally occurring phosphatide, for example lecithin; or condensation products of an alkylene oxide with fatty acids, for example polyoxyethylene stearate; or condensation products of ethylene oxide with long chain aliphatic alcohols, for example heptadecaethylene-oxycetanol; or condensation products of ethylene oxide with partial esters derived from fatty acids and a hexitol such as polyoxyethylene sorbitol monooleate; or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or α-tocopherol.

Dispersible powders and granules suitable for preparation of an aqueous suspension by the addition of water provide the compound of the invention in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solutions. Among the acceptable vehicles and solvents that may be employed are water, Ringer's solution and isotonic sodium chloride solution.

Sterile injectable preparation may also be a sterile injectable oil-in-water microemulsion where the compound of the invention is dissolved in the oily phase. For example, the active ingredient may be first dissolved in a mixture of soybean oil and lecithin. The oil solution is then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the active compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a nontoxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of the invention may also be administered in the form of a suppository for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of the invention can be employed. For purposes of this application, topical application shall include mouth washes and gargles.

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will preferably be continuous rather than intermittent throughout the dosage regimen.

The compounds of the invention may also be co-administered with other well known therapeutic agents that are selected for their particular usefulness against the condition that is being treated. The compounds may be administered simultaneously or sequentially. For example, the active compounds may be useful in combination with known anti-cancer and cytotoxic agents. Similarly, the active compounds may be useful in combination with agents that are effective in the treatment and prevention of osteoporosis, inflammation, neurofibromatosis, restinosis, and viral infections. The active compounds may also be useful in combination with inhibitors of other components of signaling pathways of cell surface growth factor receptors.

Drugs that can be co-administered to a subject being treated with a compound of the invention include antineoplastic agents selected from vinca alkaloids, epipodophyllotoxins, anthracycline antibiotics, actinomycin D, plicamycin, puromycin, gramicidin D, taxol, colchicine, cytochalasin B, emetine, maytansine, or amsacrine. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), 1996 edition (Medical Economics Company, Montvale, N.J., USA).

Radiation therapy, including x-rays or gamma rays which are delivered from either an externally applied beam or by implantation of tiny radioactive sources, may also be used in combination with a compound of the invention to treat a disease, e.g., cancer.

When a composition according to this invention is administered into a human subject, the daily dosage will normally be determined by the prescribing physician with the dosage generally varying according to the age, weight, and response of the individual patient, as well as the severity of the patient's symptoms.

Kits of the Invention

In one embodiment, a compound of the invention, materials and/or reagents required for administering the compounds of the invention may be assembled together in a kit. When the components of the kit are provided in one or more liquid solutions, the liquid solution preferably is an aqueous solution, with a sterile aqueous solution being particularly preferred.

The kit may further comprise one or more other drugs, e.g., a chemo- or radiotherapeutic agent. These normally will be a separate formulation, but may be formulated into a single pharmaceutically acceptable composition. The container means may itself be geared for administration, such as an inhalant, syringe, pipette, eye dropper, or other such like apparatus, from which the formulation may be applied to an infected area of the body, such as the lungs, or injected into an animal, or even applied to and mixed with the other components of the kit.

The compositions of these kits also may be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the agent. Kits may also comprise a compound of the invention, labeled for detecting lyases.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with a separate instrument for assisting with the injection/administration or placement of the ultimate complex composition within the body of an animal. Such an instrument may be an inhalant, syringe, pipette, forceps, measured spoon, eye dropper or any such medically approved delivery vehicle. Other instrumentation includes devices that permit the reading or monitoring of reactions or amounts of compounds or polypeptides.

The present invention is further illustrated by the following examples which should not be construed as limiting in any way. The contents of all cited references (including literature references, issued patents, published patent applications as cited throughout this application) are hereby expressly incorporated by reference.

Preparation of the Compounds of the Invention

General. All reagents are commercially available unless otherwise specified. Reagents were used as received unless otherwise specified. Proton NMR data is reported downfield from TMS. Mass spectral data (LC/MS) were obtained using a Hewlett-Packard 1100 HPLC equipped with a quaternary pump, a variable wavelength detector set at 254 nm, a YMC pro C-18 column (2×23 mm, 120A), and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA and B: 2% water in acetonitrile with 0.02% TFA. Gradient elution from 10% to 95% B over 3.5 minutes at a flow rate of 1.0 mL/min. was used with an initial hold of 0.5 minute and a final hold at 95% B of 0.5 minute. Total run time was 6.5 minutes. Purification by HPLC was performed by using a Gilson HPLC system (UV/VIS-155 detector, 215 liquid handler, 306 pumps, 819 injection valve and an 811C mixer; the column was a YMC Pro C18 (20×150 mm, Sum, 120A; the eluents were A: water with 0.1% TFA, and B: Acetonitrile with 0.1% TFA; gradient elution; flow rate was 20 mL per minute), unless otherwise indicated. Elemental analyses were obtained at Robertson Microlit Laboratories, Madison N.J. Melting points are uncorrected.

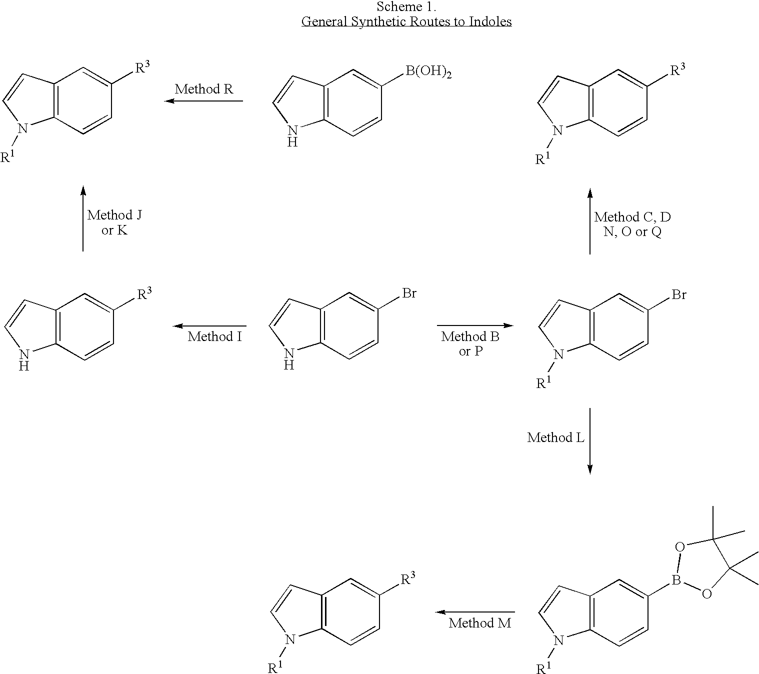

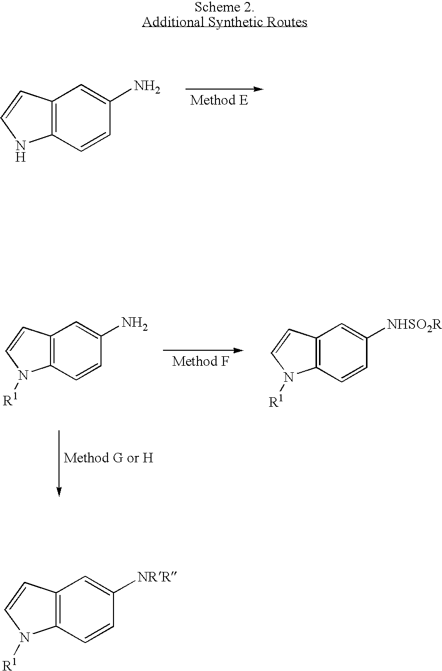

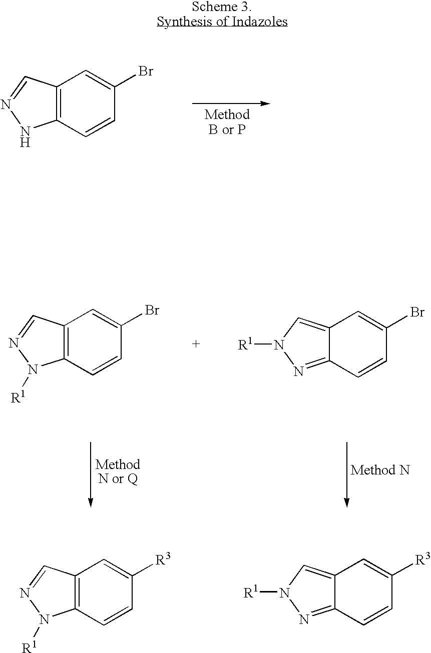

Method A. Synthesis of 4-methylpyridine-3-boronic acid (Reagent A).

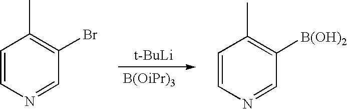

3-Bromo-4-methylpyridine (1.0 g, 5.81 mmol) was dissolved in dry tetrahydrofuran (10 mL), cooled by a dry ice-acetone bath, treated with t-butyllithium in pentane (7 mL, 1.7 M, 11.9 mmol) dropwise and stirred for 12 hour before N,N,N,N′-tetramethylethylenediamine (1.8 mL, 11.93 mmol) was added. After ½ hour, triisppropyl borate (2.75 mL, 11.92 mmol) was added dropwise. After stirring for 12 hour, the mixture was allowed to warm to room temperature and stir for another 3 hours. It was cooled by an ice water bath, treated carefully with HCl (0.5 N, 10 mL), and extracted with EtOAc (5 mL) and CH₂Cl₂/2-propanol (3:1, 5 mL). The aqueous layer was acidified with 0.5 N HCl (14 mL) to pH 8-9 and then extracted with CH₂Cl₂/2-propanol (3:1, 3×10 mL). The combined extracts were dried (sodium sulfate), filtered and concentrated to give a yellow oil. Trituration with a small amount of diethyl ether afforded a beige solid which was filtered off (322 mg, 40%) to be used for the next step without further purification. ¹H NMR (CD₃OD) δ 8.45 (s, 1H), 8.32 (d, 1H), 7.49 (d, 1H), 2.60 (s, 3H).

Method B. Exemplified by the synthesis of 5-bromo-1-(3-pyridyl)-1H-indole (Intermediate A).

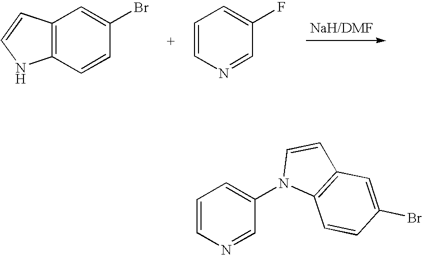

5-Bromo-1H-indole (5.0 g, 25.5 mmol) in anhydrous DMF (150 mL) was cooled to 0° C. whereby NaH (60% dispersion in mineral oil, 1.53 g, 38.3 mmol) was added in portions. Upon complete addition of NaH, the reaction mixture was allowed to warm to room temperature over 1 h. Then 3-fluoropyridine (3.71 g, 38.3 mmol) was added and the reaction mixture stirred at 100° C. overnight. The mixture was diluted with water (300 mL) and extracted with Et₂O (3×250 mL). The combined extracts were dried over Na₂SO₄, filtered and evaporated to yield a dark brown oil. Purification by flash chromatography (30% EtOAc/Hexane) provided an off-white solid (5.64 g, 81%): TLC R_{f} 0.45 (1:1 EtOAc/Hexane); HPLC Rₜ=2.85 min; ¹H-NMR (CDCl₃) δ 6.63 (d, 2H), 7.25-7.40 (m, 3H), 7.49-7.41 (m, 1H), 7.7.73-7.94 (m, 2H), 8.52 (s, 1H), 8.83 (d, 1H); LC/MS [M+1]⁺ 275.2.

Similarly prepared were the following:

5-Bromo-2,3-dimethyl-1-(3-pyridyl)-1H-indole (Intermediate B). From 5-bromo-2,3-dimethyl-1H-indole (synthesized according to Kost, A. N. et al. Chem. Heterocycl. Comp. (USSR) 1965, 1, 426-427) and 3-fluoropyridine. Yellow oil (24%). TLC R_{f} 0.22 (EtOAc/hexane 1:3); ¹H NMR (CDCl₃) δ 8.70 (broad, 2H), 7.70 (d, 1H), 7.66 (d, 1H), 7.56 (broad, 1H), 7.20 (m, 1H), 6.92 (d, 1H), 2.28 (s, 3H), 2.23 (s, 3H); LC/MS [M+1]⁺ 301.7, HPLC Rₜ=3.07 min.

5-Bromo-1-phenyl-1H-indole (Intermediate C). From 5-bromo-1H-indole and fluorobenzene. ¹H NMR (CDCl₃) δ 7.80 (s, 1H), 7.20-7.60 (m, 8H), 6.60 (d, 1H).

5-Bromo-1-(2-pyridyl)-1H-indole (intermediate D). From 5-bromo-1H-indole and 2-fluoropyridine. LC/MS [M+1]⁺ 273.1, HPLC Rₜ=4.11 min.

5-Bromo-1-(3-cyanophenyl)-1H-indole (Intermediate E). From 5-bromo-1H-indole and 3-fluorobenzonitrile. LC/MS [M+1]⁺ 297.1, HPLC Rₜ=4.18 mil.

5-Bromo-1-(2-cyanophenyl)-1H-indole (Intermediate F). From 5-bromo-1H-indole and 2-fluorobenzonitrile. LC/MS [M+1]⁺ 297.2, HPLC Rₜ=4.03 min.

5-Bromo-1-(3-pyridyl)-1H-indazole (Intermediate G). From 5-bromo-1H-indazole and 3-fluoropyridine. LC/MS [M+1]⁺ 274.3, HPLC Rₜ=2.41 min.

5-Bromo-1-(2-pyridyl)-1H-indazole (Intermediate H). From 5-bromo-1H-indazole and 2-fluoropyridine. ¹H NMR (CDCl₃) δ 8.65 (d, 1H), 8.42 (m, 1H), 8.01 (s, 1H), 7.97 (d, 1H), 7.80 (s, 1H), 7.77 (m, 1H), 7.48 (d, 1H), 7.07 (m, 1H).

5-Bromo-2-(2-pyridyl)-2H-indazole (Intermediate 1). From 5-bromo-1H-indazole and 2-fluoropyridine. ¹H NMR (CDCl₃) δ 9.02 (s, 1H), 8.50 (br, 1H), 8.24 (m, 1H), 7.88 (m, 2H), 7.60 (d, 1H), 7.33 (m, 2H).

Method C. Exemplified by the synthesis of 5-(4-methylphenyl)-1-(3-pyridyl)-1H-indole (Example 1).

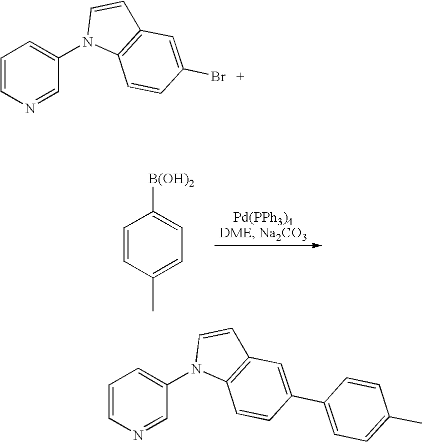

To 5-bromo-1-(3-pyridyl)-1H-indole (100 mg, 0.37 mmol) in 1,2-dimethoxyethane (3 mL, degassed under Argon) was added Pd(PPh₃)₄ (14.0 mg, 0.033 mmol). After the mixture was stirred for 5 minutes, 4-methylphenylboronic acid (60.0 mg, 0.44 mmol) and 1M Na₂CO₃ (0.93 mL) were added. The mixture was heated at 100° C. overnight, and then filtered through Celite® into CH₂Cl₂ (25 mL). The filtrate was concentrated and the residue purified by preparative TLC (1:1 EtOAc/Hex) to provide the desired product as a white solid (40.6 mg). ¹H NMR (CDCl₃) δ 8.87 (d, 1H), 8.63 (dd, 1H), 7.87 (m, 2H), 7.56 (m, 3H), 7.48 (m, 2H), 7.35 (d, 1H), 7.27 (d, 2H), 6.78 (d, 1H).

Similarly prepared were the following, characterizing data for which are shown in Tables 1 and 3 below:

### EXAMPLE 2

### 5-Phenyl-1-(3-pyridyl)-1H-indole. From 5-bromo-1-(3-pyridyl)-1H-indole and phenylboronic acid

### EXAMPLE 3

### 5-(4-Ethylphenyl)-1-(3-pyridyl)-1H-indole. From 5-bromo-1-(3-pyridyl)-1H-indole and 4-ethylphenylboronic acid

### EXAMPLE 4

### 5-(4-t-Butylphenyl)-1-(3-pyridyl)-1H-indole. From 5-bromo-1-(3-pyridyl)-1H-indole and 4-t-butylphenylboronic acid

### EXAMPLE 5

### 5-(4-Chlorophenyl)-113-pyridyl)-11H-indole. From 5-bromo-1-(3-pyridyl)-1H-indole and 4-chlorohenylboronic acid

### EXAMPLE 6

### 54-Fluorophenyl)-1-(3-pyridyl)-1H-indol). From 5-bromo-1-(3-pyridyl)-1H-indole and 4-fluorophenylboronic acid

### EXAMPLE 7

### 5-(4-Trifluoromethylphenyl)-1-(3-pyridyl)-1H-indole. From 5-bromo-1-(3-pyridyl)-1H-indole and 4-trifluoromethylphenylboronic acid

### EXAMPLE 8

### 5-(4-Methoxyphenyl)-1-(3-pyridyl)-1H-indole. From 5-bromo-1-(3-pyridyl)-1H-indole and 4-methoxyphenylboronic acid

### EXAMPLE 9

### 5-(4-Ethoxyphenyl)-113-pyridyl)-1H-indole. From 5-bromo-1-(3-pyridyl)-1H-indole and 4-ethoxyphenylboronic acid

### EXAMPLE 10

### 5-(4-Carbomethoxyphenyl)-1-(3-pyridyl)-1H-indole. From 5-bromo-1-(3-pyridyl)-1H-indole and 4-carbomethoxyphenylboronic acid

### EXAMPLE 11

### 5-(4-Carboxyphenyl)-1-(3-pyridyl)-1H-indole. From 5-bromo-1-(3-pyridyl)-1H-indole and 4-carboxyphenylboronic acid

### EXAMPLE 12

### 5-(3-Methylphenyl)-1-(3-pyridyl)-1H-indole. From 5-bromo-1-(3-pyridyl)-1H-indole and 3-methylphenylboronic acid

### EXAMPLE 13

### 5-(3-Chlorophenyl)-1-(3-pyridyl)-1H-indole. From 5-bromo-1-(3-pyridyl)-1H-indole and 3-chlorophenylboronic acid

### EXAMPLE 14

### 5-(3-Fluorophenyl)-1-(3-pyridyl)-1H-indole. From 5-bromo-1-(3-pyridyl)-1H-indole and 3-fluorophenylboronic acid

### EXAMPLE 15

### 5-(3-Trifluoromethylphenyl)-1-(3-pyridyl)-1H-indole. From 5-bromo-1-(3-pyridyl)-1H-indole and 3-trifluoromethylphenylboronic acid.

### EXAMPLE 16

### 5-(3-Methoxyphenyl)-1-(3-pyridyl)-1H-indole. From 5-bromo-1-(3-pyridyl)-1H-indole and 3-methoxyphenylboronic acid

### EXAMPLE 17

### 5-(3-Ethoxyphenyl)-1-(3-pyridyl)-1H-indole. From 5-bromo-1-(3-pyridyl)-1H-indole and 3-ethoxyphenylboronic acid

### EXAMPLE 18

### 5-(3-Nitrophenyl)-1-(3-pyridyl)-1H-indole. From 5-bromo-1-(3-pyridyl)-1H-indole and 3-nitrophenylboronic acid

### EXAMPLE 19

### 5-(3-Cyanophenyl)-1-(3-pyridyl)-1H-indole. From 5-bromo-1-(3-pyridyl)-1H-indole and 3-cyanophenylboronic acid

### EXAMPLE 20

### 5-(2-Methylphenyl)-1-(3-pyridyl)-1H-indole. From 5-bromo-1-(3-pyridyl)-1H-indole and 2-methylphenylboronic acid

### EXAMPLE 21

### 5-(2-Chlorophenyl)-1-(3-pyridyl)-1H-indole. From 5-bromo-1-(3-pyridyl)-1H-indole and 2-chlorophenylboronic acid

### EXAMPLE 22

### 5-(2-Fluorophenyl)-113-pyridyl)-1H-indole. From 5-bromo-1-(3-pyridyl)-1H-indole and 2-fluorophenylboronic acid

### EXAMPLE 23

### 5-(2-Trifluoromethylphenyl)-1-(3-pyridyl)-111-indole. From 5-bromo-1-(3-pyridyl)-1H-indole and 2-trifluoromethylphenylboronic acid

### EXAMPLE 24

### 5-(2-Methoxyphenyl)-1-(3-pyridyl)-1H-indole. From 5-bromo-1-(3-pyridyl)-1H-indole and 2-methoxyphenylboronic acid

### EXAMPLE 25

### 52-Acetylphenyl)-1-(3-pyridyl)-1H-indole. From 5-bromo-1-(3-pyridyl)-1H-indole and 2-acetylphenylboronic acid

### EXAMPLE 26

### 5-(3-Thienyl)-1-(3-pyridyl)-1H-indole. From 5-bromo-1-(3-pyridyl)-1H-indole and 3-thiopheneboronic acid

### EXAMPLE 27

### 5-(3-Furyl)-1-(3-pyridyl)-if-indole. From 5-bromo-1-(3-pyridyl)-1H-indole and 3-furanboronic acid

### EXAMPLE 28

### 1,5-bis(3-Pyridyl)-1H-indole. From 5-bromo-1-(3-pyridyl)-1H-indole and 3-pyridineboronic acid

Method D. Synthesis of 5-(4-morpholinyl)-1-(3-pyridyl)-1H-indole (Example 29)

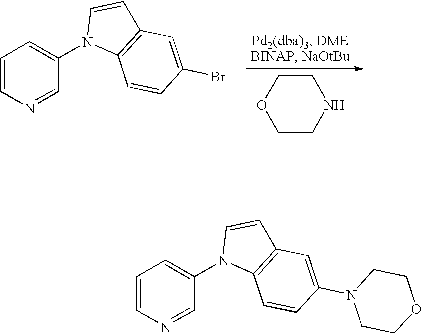

To 5-bromo-1-(3-pyridyl)-1H-indole (100 mg, 0.37 mmol) in degassed DME (2 mL) was added Pd₂(dba)₃ (10.1 mg, 0.011 mmol), BINAP (9.1 mg, 0.015 mmol), morpholine (38.3 mg, 0.44 mmol), and sodium t-butoxide (49.3 mg, 0.51 mmol). The mixture was heated at 100° C. for 4 days. It was then diluted with EtOAc and filtered. The filtrate was concentrated and the residue purified by HPLC to provide 18.2 mg of the desired product as its TFA salt. ¹HNMR (CDCl₃) 38.85 (d, 1H), 8.67 (dd, 1H), 7.89 (m, 1H), 7.75 (s, 1H), 7.57 (m, 2H), 7.42 (d, 1H), 7.36 (dd, 1H), 6.78 (d, 1H), 4.15 (dd, 4H), 3.47 (dd, 4H).

Method E. Synthesis of 5-amino-1-(3-pyridyl)-1H-indole (Intermediate J).

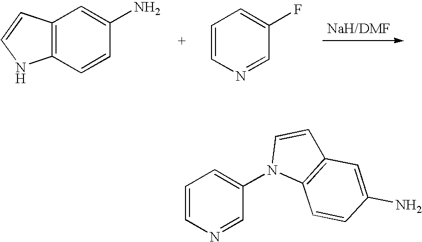

5-Amino-1H-indole (5.0 g, 37.8 mmol) in anhydrous DMF (150 mL) was cooled to 0° C. whereby NaH (60% dispersion in mineral oil, 1.82 g, 45.4 mmol) was added in portions. Upon complete addition of NaH, the reaction mixture was allowed to warm to room temperature over 1 hour. Then 3-fluoropyridine (4.41 g, 45.4 mmol) was added and the reaction mixture stirred at 100° C. overnight. The mixture was then adsorbed onto silica gel and eluted with S column volumes of EtOAc/Hexane (1:1) followed by MeOH. The MeOH eluent was concentrated to provide a dry paste which was then dissolved in a minimal amount of CH₃CN and acidified with 1 N HCl/Et₂O whereupon a white solid precipitated. The solid was filtered off and dried (7.6 g). HPLC Rₜ=0.75 min; ¹H-NMR (DMSO-d₆) δ 6.82 (d, 1H), 7.20 (d, 1H), 7.69 (r, 3H), 7.85 (s, 1H), 8.16 (d, 1H), 8.63 (d, 1H), 8.93 (s, 1H), 10.16 (br s, 2H); LC/MS [M+1]⁺ 210.2.

Method F. Exemplified by the synthesis of N-[1-(3-pyridyl)-1H-indol-5-yl]methanesulfonamide (Example 30).

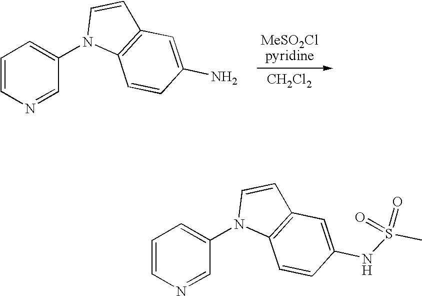

To a solution of 1-(3-pyridyl)-1H-indol-5-amine hydrochloride (100 mg, 0.41 mmol) in pyridine (3 mL) and CH₂Cl₂ (3 mL) was added methanesulfonyl chloride (69.9 mg, 0.61 mmol). The mixture was allowed to stir under argon for 16 h at room temperature. After the addition of saturated NaHCO₃ (33 mL), the mixture was extracted with CH₂Cl₂ (3×30 mL). The combined extracts were dried over Na₂SO₄, filtered, and concentrated. The solid residue was then heated in CH₃CN which was filtered while warm to provide 50.2 mg of the desired product. ¹HNMR (DMSO-d₆) δ 9.24 (s, 1H), 8.83 (s, 1H), 8.57 (d, 1H), 8.05 (d, 1H), 7.72 (s, 1H), 7.60 (m, 1H), 7.52 (m, 2H), 7.11 (d, 1H), 6.72 (d, 1H), 2.90 (s, 3H).

Similarly prepared were the following, characterizing data for which are shown in Table 2 below:

### EXAMPLE 31

### N-[1-(3-Pyridyl)-1H-indol-5-yl]ethanesulfonamide. From 1-(3-pyridyl)-1H-indol-5-amine and ethanesulfonyl chloride

### EXAMPLE 32

### N-[1-(3-Pyridyl)-1H-indol-5-yl]benzenesulfonamide., From 1-(3-pyridyl)-1H-indol-5-amine and benzenesulfonyl chloride

### EXAMPLE 33

### N-[1-(3-Pyridyl)-1H-indol-5-yl]benzylsulfonamide. From 1-(3-pyridyl)-1H-indol-5-amine and benzylsulfonyl chloride

### EXAMPLE 34

### N-[1-(3-Pyridyl)-1H-indol-5-yl]4-fluorobenzenesulfonamide. From 1-(3-pyridyl)-1H-indol-5-amine and 4-fluorobenzenesulfonyl chloride

### EXAMPLE 35

### N-[1-(3-Pyridyl)-1H-indol-5-yl]-4-cyanobenzenesulfonamide. From 1-(3-pyridyl)-1H-indol-5-amine and 4-cyanobenzenesulfonyl chloride

### EXAMPLE 36

### N-[1-(3-Pyridyl)-1H-indol-5-yl]4-methoxybenzenesulfonamide. From 1-(3-pyridyl)-1H-indol-5-amine and 4-methoxybenzenesulfonyl chloride

### EXAMPLE 37

### N-[1-(3-Pyridy11H-indol-5-yl]4-nitrobenzenesulfonamide. From 1-(3-pyridyl)-1H-indol-5-amine and 4-nitrobenzenesulfonyl chloride

### EXAMPLE 38

### N-[1-(3-Pyridyl)-1H-indol-5-yl]4-trifluoromethylbenzenesulfonamide. From 1-(3-pyridyl)-1H-indol-5-amine and 4-trifluoromethylbenzene-sulfonyl chloride

### EXAMPLE 39

### N-[1-(3-Pyridyl)-1H-indol-5-yl]A4acetylbenzenesulfonamide. From 1-(3-pyridyl)-1H-indol-5-amine and 4-acetylbenzenesulfonyl chloride

### EXAMPLE 40

### N-[1-(3-Pyridyl)-1H-indol-5-yl]-4-methylbenzenesulfonamide. From 1-(3-pyridyl)-1H-indol-5-amine and 4-methylbenzenesulfonyl chloride

### EXAMPLE 41

### N-[1-(3-Pyridyl)-1H-indol-5-yl]-4-isopropylbenzenesulfonamide. From 1-(3-pyridyl)-1H-indol-5-amine and 4-isopropylbenzenesulfonyl chloride

### EXAMPLE 42

### N-[1-(3-Pyridyl)-1H-indol-5-yl]-4-t-butylbenzenesulfonamide. From 1-(3-pyridyl)-1H-indol-5-amine and 4-t-butylbenzenesulfonyl chloride

### EXAMPLE 43

### N-[1-(3-Pyridyl)-1H-indol-5-yl]-3-fluorobenzenesulfonamide. From 1-(3-pyridyl)-1H-indol-5-amine and 3-fluorobenzenesulfonyl chloride

### EXAMPLE 44

### N-[1-(3-Pyridyl)-1H-indol-5-yl]-2-fluorobenzenesulfonamide. From 1-(3-pyridyl)-1H-indol-5-amine and 2-fluorobenzenesulfonyl chloride

### EXAMPLE 45

### N-[1-(3-Pyridyl)-1H-indol-5-yl]-2-trifluoromethylbenzenesulfonamide. From 1-(3-pyridyl)-1H-indol-5-amine and 2-trifluoromethylbenzene-sulfonyl chloride

### EXAMPLE 46

### N-[1-(3-Pyridyl)-1H-indol-5-yl]-2-metiylbenzenesulfonamide. From 1-(3-pyridyl)-1H-indol-5-amine and 2-methylbenzenesulfonyl chloride.

### EXAMPLE 47

### N-[1-(3-Pyridyl)-1H-indol-5-yl]-2-chloro-3-fluorobenzenesulfonamide. From 1-(3-pyridyl)-1H-indol-5-amine and 3-fluorobenzenesulfonyl chloride

### EXAMPLE 48

### N-[1-(3-Pyridyl)-1H-indol-5-yl]-3,4-difluorobenzenesulfonamide. From 1-(3-pyridyl)-1H-indol-5-amine and 3,4-difluorobenzenesulfonyl chloride

### EXAMPLE 49

### N-[1-(3-Pyridyl)-1H-indol-5-yl]-2,5-difluorobenzenesulfonamide. From 1-(3-pyridyl)-1H-indol-5-amine and 2,5-difluorobenzenesulfonyl chloride.

Method G. Exemplified by the synthesis of 5-(1-piperidinyl)-1-(3-pyridyl)-1H-indole (Example 50).

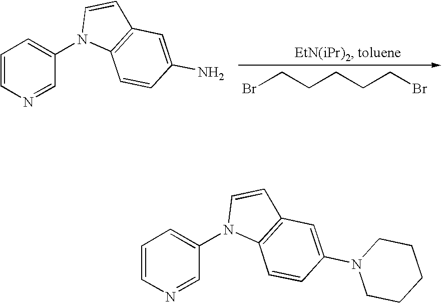

5-Amino-1-(3-pyridyl)-1H-indole dihydrochloride (100 mg, 0.35 mmol) was dissolved in dry toluene (5 mL) and treated with ethyldiisopropylamine (0.31 mL, 1.80 mmol) and 1,5-dibromopentane (0.048 mL, 0.35 mmol). The mixture was heated at reflux overnight. The solvent was decanted from an insoluble solid which was subsequently washed with ethyl acetate. The combined organic phases were washed with water, dried (sodium sulfate), filtered and concentrated to afford a colorless oil (106 mg). Purification of the oil by flash chromatography (EtOAc/hexane 1:1) gave 64 mg (65%) of a colorless oil. ¹H NMR (CDCl₃) δ 8.82 (s, 1H), 8.61 (d, 1H), 7.82 (m, 1H), 7.47 (m, 3H), 7.30 (d, 1H), 7.20 (m, 1H), 6.67 (d, 1H), 3.20 (m, 4H), 1.85 (m, 4H), 1.60 (m, 2H).

Similarly prepared was the following:

### EXAMPLE 51

### 1-(3-Pyridyl)-5-(1-pyrrolidinyl)-1H-indole. From 5-amino-1-(3-pyridyl)-1H-indole dihydrochloride and 1,4-dibromobutane. White solid (70%): ¹H NMR (CDCl₃) δ 8.82 (s, 1H), 8.55 (d, 1H), 7.80 (d, 1H), 7.43 (m, 2H), 7.24 (d, 1H), 6.85 (s, 1H), 6.70 (d, 1H), 6.60 (d, 1H), 3.37 (m, 4H), 2.05 (m, 4H)

Method H. Synthesis of 13-pyridyl)-5-phenylamino-1H-indole (Example 52).

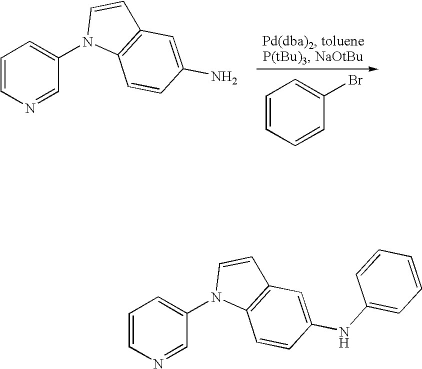

A mixture of 5-amino-1-(3-pyridyl)-1H-indole (218 mg, 1.04 mmol), bromobenzene (0.115 mL, 1.10 mmol), bis(dibenzylideneacetone)palladium (12 mg, 0.02 mmol), tri-t-butylphosphine (0.004 mL, 0.016 mmol) and sodium t-butoxide (144 mg, 1.5 mmol) in toluene (1.5 mTL) was vigorously stirred at room temperature for 3 days. The mixture was diluted with ethyl acetate, filtered and concentrated. Purification of the resulting residue by flash chromatography (EtOAc/hexane 1:1) afforded 104 mg (35%) of desired product. ¹H NMR (CDCl₃) δ 8.84 (m, 1H), 8.61 (m, 1H), 7.82 (m, 1H), 7.45 (m, 3H), 7.27 (m, 3H), 7.05 (m, 3H), 6.87 (m, 1H), 6.65 (m, 1H), 5.77 (broad, 1H).

Method L. Synthesis of 5-(3-pyridyl)-1H-indole (Intermediate K).

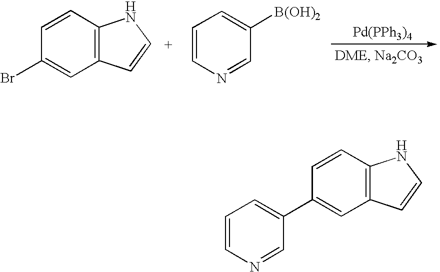

5-Bromo-1H-indole (3.0 g, 15.3 mmol) in 1,2-dimethoxyethane (30 mL) was treated with tetrakis(triphenylphosphine)palladium (1.76 g, 1.52 mmol). The mixture was stirred for 15 min. before pyridine-3-boronic acid (1.88 g, 15.3 mmol) was added, followed by 2M sodium carbonate solution (39 mL, 78 mmol). The mixture was heated at 75° C. for 5 h before the organic layer was separated. The aqueous layer was extracted with ethyl acetate. The combined organic phases were washed with a saturated sodium chloride solution, dried (sodium sulfate), filtered and concentrated. The resulting residue was purified by flash chromatography (EtOAc/hexane 1:1) to afford 0.56 g (19% yield) of a white solid. ¹H NMR (CDCl₃) δ 8.83 (s, 1H), 8.48 (m, 1H), 8.33 (broad s, 1H), 7.90 (m, 1H), 7.80 (s, 1H), 7.42 (d, 1H), 7.35 (m, 2H), 7.22 (m, 1H), 6.58 (d, 1H).

Method J. Exemplified by the synthesis of 1-(2-fluorophenyl)-5-(3-pyridyl)-1B-indole (Example 53).

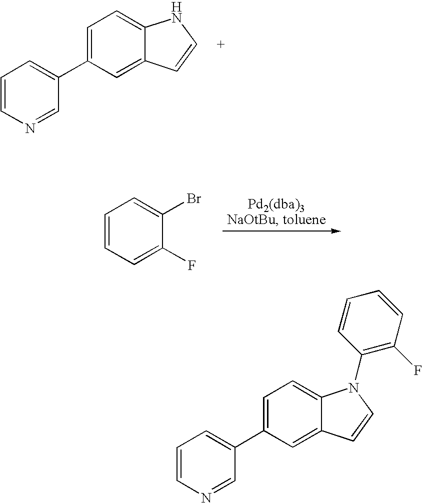

5-(3-Pyridyl)-1H-indole (50 mg, 0.26 mmol) was mixed with 1-bromo-2-fluorobenzene (0.028 mL, 0.26 mmol), 2-dicyclohexylphosphino-2′-(NN-dimethylamino)biphenyl (5 mg, 0.013 mmol), tis(dibenzylideneacetone)dipalladium (0) (12 mg, 0.013 mmol) and sodium t-butoxide (35 mg, 0.36 mmol) in toluene (2 mL) and heated at 110° C. overnight. The mixture was filtered through Celite and the filtrate concentrated to give a tan oil (45 mg). Purification of the oil by flash chromatography (EtOAc/hexane 2:3) afforded 16 mg of a pale yellow oil (22%). ¹H NMR (CDCl₃) δ 8.91 (s, 1H), 8.56 (m, 1H), 7.90 (m, 2H), 7.40 (m, 8H), 6.78 (d, 1H).

Similarly prepared were the following:

### EXAMPLE 54

### 1-(3-Cyanophenyl)-5-(3-pyridyl)-1H-indole. From 5-(3-pyridyl)-1H-indole and 3-bromobenzonitrile. Pinkish oil (9%). ¹H NMR (CD₃OD) δ 9.20 (s, 1H), 8.92 (m, 1H), 8.77 (d, 1H), 8.13 (m, 2H), 7.96 (m, 2H), 7.76 (m, 3H), 7.66 (m, 2H), 6.86 (d, 1H)

### EXAMPLE 55

### 1-(3-Fluorophenyl)-5-(3-pyridyl)-1H-indole. From 5-(3-pyridyl)-1H-indole and 3-bromo-1-fluorobenzene. Yellow oil (39%). ¹H NMR (CDCl₃) δ 8.81 (s, 1H), 8.48 (m, 1H), 7.83 (m, 2H), 7.59 (d, 1H), 7.40 (m, 2H), 7.23 (m, 4H), 7.00 (m, 1H), 6.67 (d, 1H)

Method K. Exemplified by the synthesis of 1-(2-chlorophenyl)-5-(3-pyridyl)-1H-indole (Example 56).

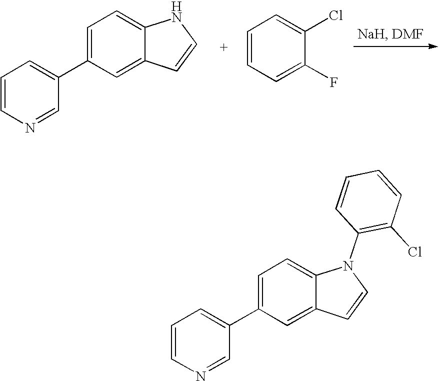

5-(3-Pyridyl)-1H-indole (50 mg, 0.26 mmol) was dissolved in dry DMF (1.5 mL), cooled by an ice water bath and treated with 60% sodium hydride (oil dispersion, 15 mg, 0.38 mmol). The mixture was stirred at room temperature for half an hour before 1-chloro-2-fluorobenzene (0.04 mL, 0.38 mmol) was added. The mixture was heated at 100° C. overnight. Ice water (7.5 mL) was carefully added. The product was extracted with ethyl acetate to give a beige oil (82 mg). Purification of the oil by flash chromatography (EtOAc/hexane 2:3) afforded 49 mg (63%) of a colorless oil. ¹H NMR (CDCl₃) δ 8.90 (s, 1H), 8.53 (m, 1H), 7.88 (m, 2H), 7.58 (m, 1H), 7.37 (m, 6H), 7.21 (d, 1H), 6.75 (d, 1H).

Similarly prepared were the following:

### EXAMPLE 57

### 1-(2-pyridyl)-5-(3-pyridyl)-1H-indole. From 5-(3-pyridyl)-1H-indole and 2-fluoropyridine. Colorless oil (96%). ¹H NMR (CDCl₃) δ 8.91 (s, 1H), 8.55 (m, 2H), 8.30 (d, 1H), 7.80 (m, 4H), 7.46 (m, 2H), 7.32 (m, 1H), 7.15 (, 11H), 6.75 (d, 1H)

### EXAMPLE 58

### 1-(2-Cyanophenyl)-5-(3-pyridyl)-1H-indole. From 5-(3-pyridyl)-1H-indole and 2-fluorobenzonitrile. White solid (86%). Mp 138-141° C.; ¹H NMR (CDCl₃) δ 8.88 (s, 1H), 8.55 (m, 1H), 7.86 (m, 3H), 7.71 (m, 1H), 7.59 (d, 1H), 7.41 (m, 5H), 6.80 (d, 1H)

### EXAMPLE 59

### 1-(3-Bromophenyl)-5-(3-pyridyl)-1H-indole. From 5-(3-pyridyl)-1H-indole and 3-bromo-1-fluorobenzene. Colorless oil (61%). ¹H NMR (CDCl₃) δ 8.92 (s, 1H), 8.58 (d, 1H), 7.90 (m, 2H), 7.65 (m, 2H), 7.42 (m, 6H), 6.75 (d, 1H)

### EXAMPLE 60

### 1-(3-Chlorophenyl)-5-(3-pyridyl)-1H-indole. From 5-(3-pyridyl)-1H-indole and 3-chloro-1-fluorobenzene. Colorless oil (63%). ¹H NMR (CDCl₃) δ 8.90 (s, 1H), 8.58 (d, 1H), 7.92 (m, 1H), 7.87 (s, 1H), 7.63 (d, 1H), 7.44 (m, 7H), 6.77 (d, 1H)

Method L. Synthesis of 1-(3-pyridyl)-5-(4,4,5,5-tetramethyl-1,3,2-dioxaborolan-2-yl)-1H-indole (Intermediate L).

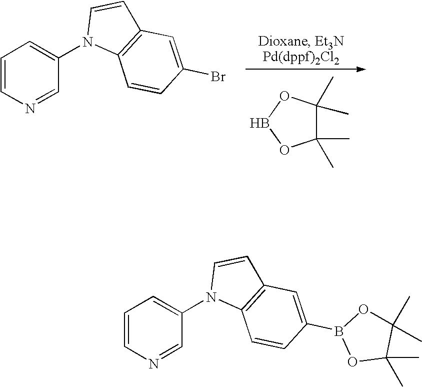

To degassed 1,4-dioxane (5 mL) was added 5-bromo-1-(3-pyridyl)-1H-indole (500 mg, 1.83 mmol), Pd(dppf)₂Cl₂ (44.8 mg, 0.055 mmol), and triethylamine (560 mg, 5.5 mmol). This was allowed to stir for 5 minutes at room temperature before 4,4,5,5-tetramethyl-1,3,2-dioxaborolane (375 mg, 2.93 mmol) was added. The mixture was heated at 80° C. for 17 h and then filtered through Celite® and a silica plug. The solvent was removed and the residue used without further purification.

Method M. Exemplified by the synthesis of 5-(2-methyl-3-pyridyl)-1-(3-pyridyl)-1H-indole (Example 61).

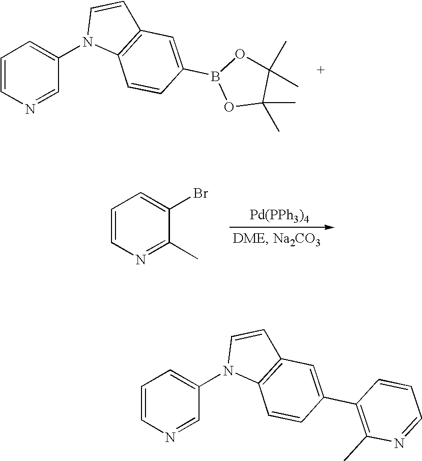

1-(3-Pyridyl)-5-(4,4,5,5-tetramethyl-1,3,2-dioxaborolan-2-yl)-1H-indole (50 mg. 0.16 mmol) was mixed with 3-bromo-2-methylpyridine (0.018 mL, 0.16 mmol) in dimethoxyethane (3 mL) and treated with tetrakis(triphenylphosphine)palladium (0) (18 mg, 0.016 mmol) and 1M sodium carbonate solution (1.5 mL, 1.5 mmol). The mixture was heated at 75° C. for 2 h and then diluted with ethyl acetate (7 mL). The organic phase was washed with saturated sodium chloride solution, dried (sodium sulfate), filtered and concentrated to give a yellow oil (62 mg). Purification of the oil by flash chromatography (EtOAc/hexane 3:1 and then 4:1) afforded 25 mg (56%) of a colorless oil. Further purification by Gilson HPLC (YMC-Packed Pro C18 Column, 150 mm×20 mm I.D.; mobile phase: 5% ACN/water (0.1% TFA) to 90% ACN/water (0.1% TFA) over 13 mm., 20 mL/min.) gave a pale yellow oil (21 mg). ¹H NMR (CDCl₃) δ 9.08 (s, 1H), 8.77 (m, 2H), 8.27 (m, 2H), 7.85 (m, 2H), 7.70 (m, 2H), 7.50 (d, 1H), 7.22 (m, 1H), 6.90 (d, 1H), 2.82 (s, 3H).

Similarly prepared were the following:

### EXAMPLE 62

### 5-(2-Pyridyl)-1-(3-pyridyl)-1-H-indole. From 1-(3-pyridyl)-5-(4,4,5,5-tetramethyl-1,3,2-dioxaborolan-2-yl)-1H-indole and 2-bromopyridine. Colorless oil (31%). ¹H NMR (CDCl₃) δ 8.80 (d, 1H), 8.63 (s, 1H), 8.59 (s, 1H), 8.28 (d, 1H), 7.77 (m, 4H), 7.55 (m, 1H), 7.43 (m, 1H), 7.30 (m, 1H), 7.20 (m, 1H), 6.78 (d, 1H).

### EXAMPLE 63

### 1-(3-Pyridyl)-5-(4-pyridyl)-1H-indole. From 1-(3-pyridyl)-5-(4,4,5,5-tetramethyl-1,3,2-dioxaborolan-2-yl)-1H-indole and 4-bromopyridine. White solid (50%).

¹H NMR (CDCl₃) δ 8.79 (d, 1H), 8.57 (m, 3H), 7.90 (s, 1H), 7.79 (m, 1H), 7.45 (m, 5H), 7.32 (d, 1H), 6.74 (d, 1H).

### EXAMPLE 64

### 5-(2-Methyl-5-pyridyl)-1-(3-pyridyl)-1H-indole. From 1-(3-pyridyl)-5-(4,4,5,5-tetramethyl-1,3,2-dioxaborolan-2-yl)-1H-indole and 5-bromo-2-methylpyridine. Pale yellow oil (25%). ¹H NMR (CDCl₃) δ 9.05 (m, 2H), 8.75 (d, 1H), 8.50 (d, 1H), 8.27 (d, 1H), 7.99 (s, 1H), 7.88 (m, 1H), 7.70 (m, 2H), 7.52 (m, 2H), 6.90 (d, 1H), 2.86 (s, 3H).

### EXAMPLE 65

### 5-(S-Cyano-3-pyridyl)-1-(3-pyridyl)-1H-indole. From 1-(3-pyridyl)-5-(4,4,5,5-tetramethyl-1,3,2-dioxaborolan-2-yl)-1H-indole and 3-bromo-5-cyanopyridine. Pale yellow solid (33%). ¹H NMR (CDCl₃) δ 9.10 (s, 1H), 8.88 (d, 1H), 8.82 (s, 1H), 8.68 (d, 1H), 8.20 (d, 1H), 7.95 (m, 2H), 7.60 (m, 2H), 7.45 (m, 2H), 6.83 (d, 1H).

### EXAMPLE 66

### 5-(4-Methyl-3-pyridyl)-1-(3-pyridyl)-1H-indole. From 1-(3-pyridyl)-5-(4,4,5,5-tetramethyl-1,3,2-dioxaborolan-2-yl)-1H-indole and 3-bromo-4-methylpyridine.

Method N. Exemplified by the synthesis of 2,3-dimethyl-1,5-bis(3-pyridyl)-1H-indole (Example 67).

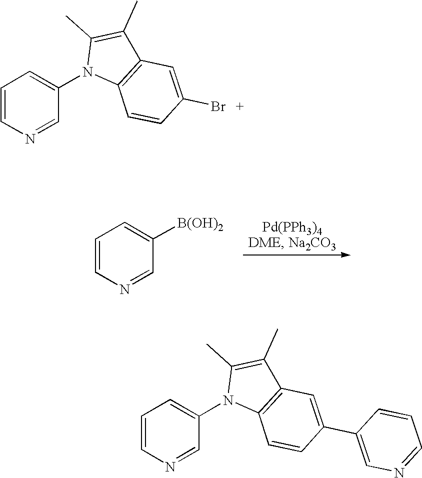

5-Bromo-2,3-dimethyl-1-(3-pyridyl)-1H-indole (73 mg, 0.24 mmol) was dissolved in 1,2-dimethoxyethane (2 mL) and treated with pyridine-3-boronic acid (30 mg. 0.24 mmol), tetrakis(triphenylphosphine)palladium (28 mg, 0.024 mmol) and 2M sodium carbonate solution (1.2 mL, 2.4 mmol). The mixture was heated at 75° C. for 3 h and then extracted with ethyl acetate to give a yellow oil (97 mg). Purification of the oil by flash chromatography (EtOAc/hexane 2:1) afforded 41 mg (56%) of an oil. Further purification by Gilson HPLC (YMC-Packed Pro C18 Column, 150 mm×20 mm I.D.; mobile phase: 40% ACN/water (0.1% TFA) to 90% ACN/water (0.1% TFA) over 13 min., 20 mL/min.) yielded 22 mg of a beige oil. ¹H NMR (CD₃OD) δ9.21 (s, 1H), 8.97 (d, 1H), 8.81 (m, 3H), 8.23 (m, 1H), 8.14 (m, 1H), 8.02 (s, 1H), 7.91 (m, 1H), 7.57 (d, 1H), 7.30 (d, 1H), 2.40 (s, 3H), 2.30 (s, 3H).

Similarly prepared were the following:

### EXAMPLE 68

### 5-(2-Chlorophenyl)-2,3-dimethyl-1-(3-pyridyl)-1H-indole. From 5-bromo-2,3-dimethyl-1-(3-pyridyl)-1H-indole and 2-chlorophenylboronic acid. Yellow oil (13%). ¹H NMR (CD₃OD) δ 8.82 (m, 2H), 8.28 (m, 1H), 7.94 (m, 1H), 7.52 (s, 1H), 7.47 (m, 1H), 7.34 (m, 3H), 7.18 (s, 2H), 2.32 (s, 3H), 2.30 (s, 3H)

### EXAMPLE 69

### 1-Phenyl-5-(3-pyridyl)-1H-indole. From 5-bromo-1-phenyl-1H-indole and pyridine-3-boronic acid. Colorless oil (39%). ¹H NMR (CDCl₃) δ 8.94 (s, 1H), 8.58 (m, . 1H), 7.90 (m, 2H), 7.64 (d, 1H), 7.50 (m, 4H), 7.40 (m, 4H), 6.78 (d, 1H).

### EXAMPLE 70

### 5-(4-Methyl-3-pyridyl)-1-(2-pyridyl)-1H-indole. From 5-bromo-1-(2-pyridy)-1H-indole and 4-methylpyridine-3-boronic acid. Beige oil (42%). ¹H NMR (CDCl₃) δ 8.59 (m, 1H), 8.51 (s, 1H), 8.43 (d, 1H), 8.32 (d, 1H), 7.83 (m, 1H), 7.77 (m, 1H), 7.59 (s, 1H), 7.50 (d, 1H), 7.20 (m, 3H), 6.77 (d, 1H), 2.35 (s, 3H).

### EXAMPLE 71

### 1-(3-Cyanophenyl)-5-(4-methyl-3-pyridyl)-1H-indole. From 5-bromo-1-(3-cyanophenyl)-1H-indole and 4-methylpyridine-3-boronic acid. Pale yellow solid (46%). ¹H NMR (CDCl₃) δ 8.50 (s, 1H), 8.43 (d, 1H), 7.83 (s, 1H), 7.80 (m, 1H), 7.62 (m, 4H), 7.38 (d, 1H), 7.20 (m, 2H), 6.79 (d, 1H), 2.34 (s, 3H).

### EXAMPLE 72

### 1-(2-Cyanophenyl)-5-(4-methyl-3-pyridyl)-1H-indole. From 5-bromo-1-(2-cyanophenyl)-1H-indole and 4-methylpyridine-3-boronic acid. Beige solid (50%). ¹H NMR (CDCl₃) δ 8.52 (s, 1H), 8.43 (d, 1H), 7.88 (d, 1H), 7.77 (m, 1H), 7.62 (m, 2H), 7.50 (m, 2H), 7.41 (d, 1H), 7.20 (m, 2H), 6.80 (d, 1H), 2.33 (s, 3H).

### EXAMPLE 73

### 1,5-bis(3-pyridyl)-1H-indazole. From 5-bromo-1-(3-pyridyl)-1H-indazole and pyridine-3-boronic acid. White solid (60%). ¹H NMR (CDCl₃) δ 9.10 (d, 1H), 8.90 (d, 1H), 8.62 (m, 2H), 8.30 (s, 1H), 8.09 (m, 1H), 8.00 (s, 1H), 7.93 (m, 1H), 7.83 (d, 1H), 7.68 (d, 1H), 7.49 (m, 1H), 7.40 (m, 1H).

### EXAMPLE 74

### 5-(4-Fluorophenyl)-1-(3-pyridyl)-1H-indazole. From 5-bromo-1-(3-pyridyl)-1H-indazole and 4-fluorophenylboronic acid. White solid (43%). ¹H NMR (CDCl₃) δ 9.02 (s, 1H), 8.54 (m, 1H), 8.21 (s, 1H), 8.04 (m, 1H), 7.85 (s, 1H), 7.73 (d, 1H), 7.60 (d, 1H), 7.26 (m, 3H), 7.07 (m, 2H).

### EXAMPLE 75

### 5-(4-Methyl-3-pyridyl)-1-(2-pyridyl)-1H-indazole. From 5-bromo-1-(2-pyridyl)-1H-indazole and 4-methylpyridine-3-boronic acid. Beige oil (43%). ¹H NMR (CDCl₃) δ 8.90 (d, 1H), 8.53 (m, 2H), 8.47 (d, 1H), 8.22 (s, 1H), 8.05 (d, 1H), 7.82 (m, 1H), 7.68 (s, 1H), 7.44 (d, 1H), 7.19 (m, 2H), 2.31 (s, 3H).

### EXAMPLE 76

### 5-(4-Methyl-3-pyridyl)-2-(2-pyridyl)-2H-indazole. From 5-bromo-2-(2-pyridyl)-2H-indazole and 4-methylpyridine-3-boronic acid. Beige oil (32%). ¹H NMR (CDCl₃) δ 9.18 (s, 1H), 8.53 (m, 2H), 8.47 (d, 1H), 8.30 (d, 1H), 7.92 (m, 1H), 7.81 (d, 1H), 7.65 (s, 1H), 7.27 (m, 3H), 2.35 (s, 3H).

Method O. Synthesis of 3-[5-(4-methyl-3-pyridyl)-1H-indol-1-yl]benzamide (Example 77)

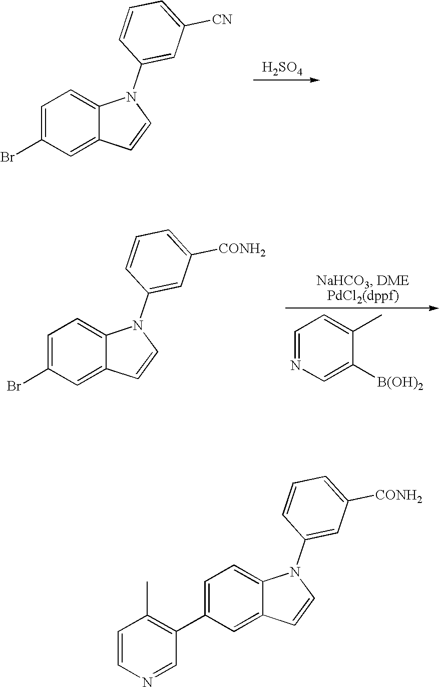

Step 1. 5-Bromo-1-(3-cyanophenyl)-1H-indole (100 mg, 0.34 mmol) was stirred in conc. H₂SO₄ at room temperature overnight. Then the mixture was carefully added to cold sodium carbonate solution until pH 4-5. Extraction with CH₂Cl₂ and also CH₂Cl₂/2-propanol (4:1) gave 104 mg (98%) of 3-(5-bromo-1H-indol-1-yl)benzamide as a yellow solid. A portion of the solid was purified by Gilson HPLC (YMC-Packed Pro C18 Column, 150×20 mm I.D.; mobile phase: 70-90% ACN/water (0.1% TFA) over 13 min., 20 mL/min.) to afford a white solid. ¹H NMR (CD₃OD) δ 8.02 (m, 1H), 7.89 (m, 1H), 7.70 (m, 3H), 7.53 (d, 1H), 7.47 (d, 1H), 7.30 (d, 1H), 6.66 (d, 1H); LC/MS [M+1]⁺ 315.0 (M+H⁺), HPLC Rₜ=2.89 ml.

Step 2. A solution of 3-(5-bromo-1H-indol-1-yl)benzamide (50 mg, 0.16 mmol) in 1,2-dimethoxyethane (2 mL) and water (1 mL) was degassed for five minutes before sodium bicarbonate (53 mg, 0.63 mmol), 4-methylpyridine-3-boronic acid (33 mg, 0.24 mmol) and 1,1′-bis(diphenylphosphino)ferrocene dichloropalladium (II) complex with dichloromethane (13 mg, 0.016 mmol) were added. The mixture was heated at reflux overnight. Extraction with ethyl acetate gave a dark oil (56 mg). Purification of the oil by flash chromatography (2% 2M NH₃/MeOH in EtOAc) afforded 12 mg (23%) of a beige solid. ¹H NMR (CDCl₃) δ 8.42 (s, 1H), 8.38 (m, 1H), 8.00 (s, 1H), 7.72 (d, 1H), 7.63 (d, 1H), 7.57 (m, 3H), 7.38 (d, 1H), 7.14 (m, 2H), 6.67 (d, 1H), 2.27 (s, 3H).

Method P. Exemplified by the synthesis of 5-bromo-1-(4-methyl-3-pyridyl)-1H-indazole (Intermediate M).

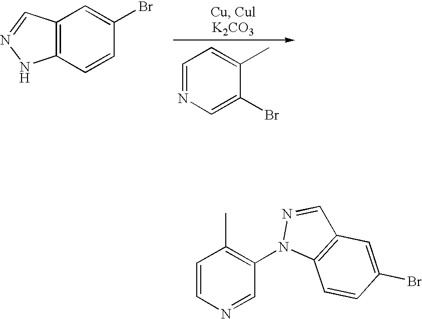

5-Bromo-1H-indazole (465 mg, 2.36 mmol; prepared according to Dell'Erba, C. et al. Tetrahedron 1994, 50, 3529-3536) was mixed with copper (423 mg, 6.66 mol), cuprous iodide (63 mg, 0.33 mmol), potassium carbonate (1.3 g, 9.41 mmol) and 3-bromo-4-methylpyridine (1.2 mL), and then heated at 200° C. overnight. Dichloromethane was added and the mixture was filtered through Celite. The dark mass obtained after concentrating the filtrate was purified by flash chromatography (EtOAc/hexane 2:3) to give a beige solid (30 mg, 4%). LCMS [M+1]⁺ 288.2, HPLC Rₜ=2.37 min.

Similarly prepared were the following, characterizing data for which are shown in Table 4 below:

### EXAMPLE 78

### 5-Bromo-1-(4-methyl-3-pyridyl)-1H-indole. From 5-bromo-1H-indole and 3-bromo-4-methylpyridine

### EXAMPLE 79

### 1-(4-Methyl-3-pyridyl)-1H-indole-5-carbonitrile: From 1H-indole-5-carbonitrile and 3-bromo-4-methylpyridine

### EXAMPLE 80

### Benzyl 1-(4-methyl-3-pyridyl)-1H-indol-5-yl ether: From Benzyl 1H-indol-5-yl ether and 3-bromo-4-methylpyridine

5-Bromo-1-(2-methyl-3-pyridyl)-1H-indole (Intermediate N). From 5-bromo-1H-indole and 3-bromo-2-methylpyridine.

5-Bromo-1-(2-methyl-5-pyridyl)-1H-indole (Intermediate 0). From 5-bromo-1H-indole and 5-bromo-2-methylpyridine.

Method Q. Exemplified by the synthesis of 1-(4-methyl-3-pyridyl)-5-(3-pyridyl)-1H-indazole bis(trifluoroacetate) (Example 81)

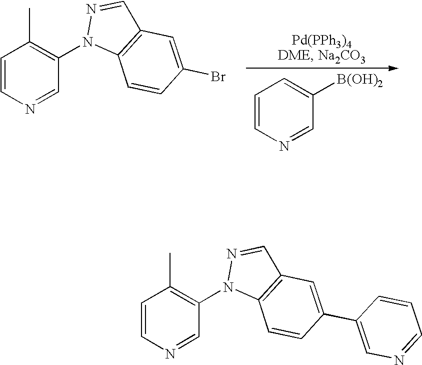

To 5-bromo-1-(4-methyl-3-pyridyl)-1H-indazole (30 mg, 0.104 mmol) in 1,2-dimethoxyethane (1 mL) was added pyridine-3-boronic acid (26 mg, 0.212 mmol), tetrakis(triphenylphosphine)palladium (24 mg, 0.021 mmol) and 2M sodium carbonate solution (0.5 mL, 1.0 mmol). The mixture was heated at 85° C. overnight. Extraction with !o dichloromethane gave a brown residue (40 mg) which was purified by Gilson HPLC (YMC-Packed Pro C18 Column, 100×20 mm I.D.; mobile phase: 10-95% ACN/water (0.1% TFA) over 9 min., 20 mL/min.) to afford 7 mg (13%) of a colorless oil. ¹H NMR (CD₃OD) δ 9.24 (s, 1H), 8.95 (m, 1H), 8.82 (m, 2H), 8.74 (m, 1H), 8.53 (s, M1), 8.41 (s, 1H), 8.16 (m, 1H), 7.96 (d, 1H), 7.85 (m, 1H), 7.60 (d, 1H), 2.38 (s, 3H).

Similarly prepared were the following:

### EXAMPLE 82

### 1-(4-Methyl-3-pyridyl)-5-(3-pyridyl)-1H-indole. From 5-bromo-1-(4-methyl-3-pyridyl)-1H-indole and pyridine-3-boronic acid. Beige gel (27%); ¹H NMR (CD₃OD) δ 9.20 (s, 11), 8.85 (m, 2H), 8.79 (m, 2H), 8.20 (s, 1H), 8.15 (m, 1H), 8.02 (d, 1H), 7.67 (d, 1H), 7.56 (d, 1H), 7.33 (d, 1H), 6.97 (d, 1H), 2.34 (s, 3H)

### EXAMPLE 83

### 1-(4-Methyl-3-pyridyl)-5-(4-pyridyl)-1H-indole. From 5-bromo-1-(4-methyl-3-pyridyl)-1H-indole and pyridine-4-boronic acid. Yellow gel (25%); ¹H NMR (CD₃OD) δ 8.79 (m, 4H), 8.43 (m, 3H), 7.94 (m, 1 μl), 7.88 (m, 1H), 7.59 (d, 1H), 7.35 (d, 1H), 7.00 (d, 1H), 2.30 (s, 3H)

### EXAMPLE 84

### 1,5-bis(4-Methyl-3-pyridyl)-1H-indole. From 5-bromo-1-(4-methyl-3-pyridyl)-1H-indole and 4-methylpyridine-3-boronic acid. Colorless gel (33%); ¹H NMR (CD₃OD) δ 8.91 (broad, 1H), 8.80 (broad, 1H), 8.73 (s, 1H), 8.69 (d, 1H), 8.02 (m, 2H), 7.83 (s, 1H), 7.58 (d, 1H), 7.30 (m, 2H), 6.94 (d, 1H), 2.63 (s, 3H), 2.38 (s, 3H)

### EXAMPLE 85

### 5-(3-Furyl)-1-(4-methyl-3-pyridyl)-1H-indole). From 5-bromo-1-(4-methyl-3-pyridyl)-1H-indole and furan-3-boronic acid. Beige gel (19%); ¹H NMR (CD₃OD) δ 8.82 (broad, 1H), 8.73 (broad, 1H), 7.94 (m, 1H), 7.84 (m, 2H), 7.53 (d, 1H), 7.42 (d, 11), 7.39 (d, 1H), 7.07 (d, 1H), 6.80 (m, 2H), 2.32 (s, 3H)

### EXAMPLE 86

### 1-(4-Methyl-3-pyridyl)-5-(3-thienyl)-1H-indole. From 5-bromo-1-(4-methyl-3-pyridyl)-1H-indole and thiophene-3-boronic acid. Yellow gel (10%); ¹H NMR (CD₃OD) δ 8.78 (m, 2H), 7.95 (m, 2H), 7.47 (m, 5H), 7.10 (d, 1H), 6.81 (d, 1H), 2.30 (s, 3H)

### EXAMPLE 87

### 1-(4-Methyl-3-pyridyl)-5-phenyl-1H-indole. From 5-bromo-1-(4-methyl-3-pyridyl)-1H-indole and phenylboronic acid. Yellow gel (14%); ¹H NMR (CD₃OD) δ 8.75 (m, 1H), 8.68 (m, 1H), 7.92 (s, 1H), 7.85 (d, 1H), 7.63 (d, 2H), 7.42 (m, 4H), 7.27 (m, 1H), 7.13 (d, 1H), 6.84 (d, 1H), 2.30 (s, 3H)

### EXAMPLE 88

### 5-(2-Fluorophenyl)-1-(4-methyl-3-pyridyl)-1H-indole. From 5-bromo-1-(4-methyl-3-pyridyl)-1H-indole and 2-fluorophenylboronic acid. Beige gel (28%); ¹H NMR (CD₃OD) δ 8.43 (m, 2H), 7.75 (s, 1H), 7.44 (m, 2H), 7.20 (m, 5H), 6.95 (d, 1H), 6.70 (d, 1H), 2.08 (s, 3H)

### EXAMPLE 89

### 5-(2-Cyanophenyl)-1-(4-methyl-3-pyridyl)-1H-indole. From 5-bromo-1-(4-methyl-3-pyridyl)-1H-indole and 2-cyanophenylboronic acid. Beige gel (5%); ¹H NMR (CD₃OD) δ 8.43 (m, 2H), 7.80 (d, 1H), 7.74 (d, 1H), 7.45 (m, 6H), 7.01 (d, 1H), 6.75 (d, 1H), 2.10 (s, 3H)

### EXAMPLE 90

### 5-(2-Chlorophenyl)-1-(4-methyl-3-pyridyl)-1H-indole. From 5-bromo-1-(4-methyl-3-pyridyl)-1H-indole and 2-chlorophenylboronic acid. Beige gel (7%); ¹H NMR (CD₃OD) δ 8.44 (d, 2H), 7.60 (s, 1H), 7.30 (m, 7H), 6.94 (d, 1H), 6.68 (d, 1H), 2.09 (s, 3H)

### EXAMPLE 91

### 5-(3-Fluorophenyl)-1-(4-methyl-3-pyridyl)-1H-indole. From 5-bromo-1-(4-methyl-3-pyridyl)-1H-indole and 3-fluorophenylboronic acid. Beige gel (43%); ¹H NMR (CD₃OD) δ 8.42 (broad, 2H), 7.82 (s, 1H), 7.46 (d, 1H), 7.34 (m, 5H), 6.94 (m, 2H), 6.72 (d, 1H), 2.06 (s, 3H)

### EXAMPLE 92

### 5-(3-Chlorophenyl)-1-(4-methyl-3-pyridyl)-1H-indole. From 5-bromo-1-(4-methyl-3-pyridyl)-1H-indole and 3-chlorophenylboronic acid. Beige gel (10%); ¹H NMR (CD₃OD) δ 8.65 (m, 2H), 7.82 (s, 1H), 7.78 (m, 1H), 7.52 (s, 1H), 7.45 (d, 1H), 7.37 (d, 1H), 7.30 (d, 2H), 7.19 (d, 1H), 7.04 (d, 1H), 6.75 (d, 1H), 2.20 (s, 3H)

### EXAMPLE 93

### 1-(4-Methyl-3-pyridyl)-5-(3-nitrophenyl)-1H-indole. From 5-bromo-1-(4 methyl-3-pyridyl)-1H-indole and 3-nitrophenylboronic acid. Yellow gel (26%); ¹H NMR (CD₃OD) δ 8.82 (broad, 2H), 8.48 (d, 11), 8.18 (d, 1 μl), 8.02 (m, 3H), 7.67 (m, 11), 7.57 (d, 1H), 7.48 (d, 1H), 7.21 (d, 1H), 6.90 (d, 1H), 2.36 (s, 3H)

### EXAMPLE 94

### 5-(4-Fluorophenyl)-1-(4-Methyl-3-pyridyl)-1B-indole. From 5-bromo-1-(4-methyl-3-pyridyl)-1H-indole and 4-fluorophenylboronic acid. Beige powder (41%); Mp 120-122° C.; ¹H NMR (CDCl₃) δ 8.68 (broad, 2H), 7.89 (s, 1H), 7.61 (m, 2H), 7.40 (d, 2H), 7.14 (m, 4H), 6.79 (d, 1H), 2.18 (s, 3H)

### EXAMPLE 95

### 5-(4-Cyanophenyl)-1-(4-methyl-3-pyridyl)-1H-indole. From 5-bromo-1-(4-methyl-3-pyridyl)-1H-indole and 4-cyanophenylboronic acid. Yellow gel (13%); ¹H NMR (CD₃OD) δ 8.80 (m, 2H), 8.02 (s, 1H), 7.97 (m, 1H), 7.83 (d, 2H), 7.77 (d, 2H), 7.55 (d, 1H), 7.46 (d, 1H), 7.20 (d, 1H), 6.89 (d, 1H), 2.32 (s, 3H)

### EXAMPLE 96

### 5-(4-Chlorophenyl)-1-(4-methyl-3-pyridyl)-1H-indole. From 5-bromo-1-(4-methyl-3-pyridyl)-1H-indole and 4-chlorophenylboronic acid. Yellow gel (9%); ¹H NMR (CD₃OD) δ 8.83 (m, 1H), 8.74 (m, 1H), 7.95 (m, 2H), 7.62 (m, 2H), 7.44 (m, 4H), 7.15 (d, 1H), 6.83 (d, 1H), 2.32 (s, 3H)

### EXAMPLE 97

### 1-(4-Methyl-3-pyridyl)-5-(4-trifluoromethylphenyl)-1H-indole. From 5-bromo-1-(4-methyl-3-pyridyl)-1H-indole and 4-trifluoromethylphenyl-boronic acid. Beige gel (21%); ¹H NMR (CD₃OD) δ 8.81 (m, 2H), 8.00 (m, 2H), 7.82 (d, 2H), 7.71 (d, 2H), 7.56 (d, 1H), 7.44 (d, 1H), 7.20 (d, 1H), 6.88 (d, 1H), 2.34 (s, 3H)

### EXAMPLE 98

### 5-(4-Methoxyphenyl)-1-(4-methyl-3-pyridyl)-1H-indole. From 5-bromo-1-(4-methyl-3-pyridyl)-1H-indole and 4-methoxyphenyl-boronic acid. Yellow gel (29%); ¹H NMR (CD₃OD) δ 8.83 (broad, 1H), 8.73 (broad, 1H), 7.95 (d, 1H), 7.85 (s, 1H), 7.56 (d, 2H), 7.45 (d, 1H), 7.40 (d, 1H), 7.10 (d, 1H), 6.99 (d, 2H), 6.82 (d, 1H), 3.81 (s, 3H), 2.35 (s, 3H)

### EXAMPLE 99

### 1-(2-Methyl-3-pyridyl)-5-(3-pyridyl)-1H-indole. From 5-bromo-1-(2-methyl-3-pyridyl)-1H-indole and pyridine-3-boronic acid. Beige gel (96%); ¹H NMR (CDCl₃) δ 8.83 (m, 1H), 8.59 (m, 1H), 8.50 (m, 1H), 7.85 (m, 2H), 7.60 (m, 1H), 7.30 (m, 3H), 7.15 (m, 1H), 7.04 (d, 1H), 6.72 (d, 1H), 2.30 (s, 3H)

### EXAMPLE 100

### 5-(4-Fluorophenyl)-1-(2-methyl-3-pyridyl)-1H-indole. From 5-bromo-1-(2-methyl-3-pyridyl)-1H-indole and 4-fluorophenylboronic acid. Beige gel (41%); ¹H NMR (CDCl₃) δ 8.59 (d, 1H), 7.80 (s, 1H), 7.64 (d, 1H), 7.52 (m, 2H), 7.30 (m, 2H), 7.07 (m, 4H), 6.71 (d, 111, 2.30 (s, 3H)

### EXAMPLE 101

### 5-(3-Cyanophenyl)-1-(2-methyl-5-pyridyl)-1H-indole. From 5-bromo-1-(2-methyl-5-pyridyl)-1H-indole and 3-cyanophenylboronic acid. White powder (7%); ¹H NMR (CDCl₃) δ 8.70 (s, 1H), 7.90 (m, 3H), 7.74 (m, 1H), 7.57 (m, 3H), 7.40 (m, 3H), 6.79 (d, 1H), 2.66 (s, 3H)

### EXAMPLE 102

### 1-(2-Methyl-5-pyridyl)-5-(3-pyridyl)-1-indole. From 5-bromo-1-(2 methyl-5-pyridyl)-1H-indole and pyridine-3-boronic acid. Clear gel (13%); ¹H NMR (CD₃OD) δ 9.21 (s, 1H), 8.97 (m, 2H), 8.80 (d, 1H), 8.50 (d, 1H), 8.15 (m, 2H), 7.90 (d, 1H), 7.80 (d, 1H), 7.73 (m, 2H), 6.96 (d, 1H), 2.80 (s, 3H).

### EXAMPLE 103

### 3-[1-(4-Methyl-3-pyridyl)-1H-indol-5-yl]benzonitrile. From 5-bromo-1-(4-methyl-3-pyridyl)-1H-indole and 3-cyanophenylboronic acid. ¹H NMR (CD₃OD, TFA salt) δ 8.80 (s, 1H), 8.70 (d, 1H), 8.00 (m, 3H), 7.91 (d, 1H), 7.65 (m, 2H), 7.53 (d, 1H), 7.45 (d, 1H), 7.19 (d, 1H), 6.88 (d, 1H), 2.30 (s, 3H)

Method R. Synthesis of 1-(4-methyl-3-pyridyl)-5-(3-pyrimidyl)-1H-indole (Example 104).

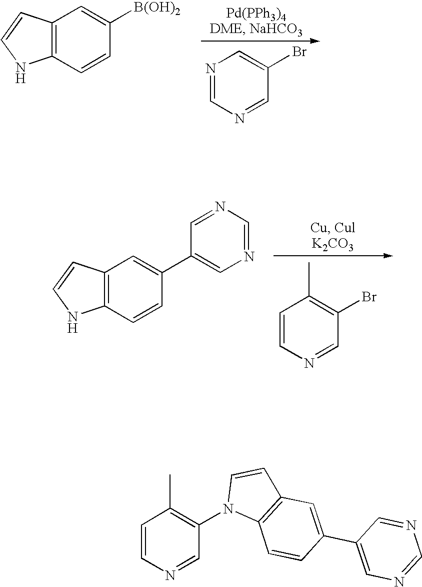

Step 1. A mixture of 5-indolylboronic acid (2 g, 12.4 mmol), 5-bromopyrimidine (1.85 g, 11.3 mmol), sodium bicarbonate (2.85 g, 33.9 mmol), tetrakis(triphenylphosphine) palladium (0.66 mg, 0.57 mmol), 1,2-dimethoxyethane (100 mL) and water (50 mL) were heated at reflux for 6 h. The reaction mixture was diluted with dichloromethane and washed with water (2×) and brine. The organic layer was dried (sodium sulfate), filtered and concentrated in vacuo to give a crude product. Purification by flash chromatography (EtOAc/hexane 1:1) afforded 1.761 g (80%) of 5-(5-pyrimidyl)-1H-indole: LCMS [M+1]⁺ 196, HPLC Rₜ=1.89 min.

Step 2. A mixture 5-(5-pyrimidyl)-1H-indole (1.6 g, 8.196 mmol), copper(I) iodide (0.19 g, 0574 mmol), copper (0.73 g, 11.74 mmol), potassium carbonate (2.83 g, 20.5 mmol) and 3-bromo-4-methylpyridine (8 mL) was stirred at 200° C. under argon for 4 h. The mixture was cooled to room temperature and diluted with dichloromethane. Filtration followed by concentration of the filtrate gave a brown oil. Purification by flash chromatography (EtOAc/hexane 4:1) afforded 1.9 g (82%) of 1-(4-methyl-3-pyridyl)-5-(5-pyrimidyl)-1H-indole. Anal. Calcd for C₁₈H₁₄N₄: C, 75.50; H, 4.93; N, 19.57. Found: C, 75.41; H, 4.85; N, 19.70.

The following compounds, as illustrative examples, can be made from commercially available 4-bromo-1H-indole and 6-bromo-1H-indole using the methods depicted in Scheme 1 and the appropriate reagents:

### EXAMPLE 105

### 4-Phenyl-1-(3-pyridyl)-1H-indole

### EXAMPLE 106

### 6-Phenyl-1-(3-pyridyl)-1H-indole

### EXAMPLE 107

### 4-(4-t-Butylphenyl)-1-(3-pyridyl)-1H-indole

### EXAMPLE 108

### 6-(4-Chlorophenyl)-1-(3-pyridyl)-1H-indole

### EXAMPLE 109

### 4-(4-Fluorophenyl)-1-(3-pyridyl)-1H-indole

### EXAMPLE 110

### 6-(4-Trifluoromethylphenyl)-1-(3-pyridyl)-1H-indole

### EXAMPLE 111

### 4-(4-Methoxyphenyl)-1-(3-pyridyl)-1H-indole

### EXAMPLE 112

### 6-(4-Ethoxyphenyl)-1-(3-pyridyl)-1H-indole

### EXAMPLE 113

### 4-(3-Furyl)-1-(3-pyridyl)-1H-indole

### EXAMPLE 114

### 6-(3-Methylphenyl)-1-(3-pyridyl)-1H-indole

### EXAMPLE 115

### 4-(3-Chlorophenyl)-1-(3-pyridyl)-1H-indole

### EXAMPLE 116

### 6-(3-Fluorophenyl)-1-(3-pyridyl)-1H-indole

### EXAMPLE 117

### 4-(3-Trifluoromethylphenyl)-1-(3-pyridyl)-1H-indole

### EXAMPLE 118

### 6-(3-Methoxyphenyl)-1-(3-pyridyl)-1H-indole

### EXAMPLE 119

### 4-(3-Ethoxyphenyl)-1-(3-pyridyl)-1H-indole

### EXAMPLE 120

### 6-(3-Nitrophenyl)-1-(3-pyridyl)-1H-indole

### EXAMPLE 121

### 4-(3-Cyanophenyl)-1-(3-pyridyl)-1H-indole

### EXAMPLE 122

### 6-(2-Methylphenyl)-1-(3-pyridyl)-1H-indole

### EXAMPLE 123

### 4-(2-Chlorophenyl)-1-(3-pyridyl)-1H-indole

### EXAMPLE 124

### 6-(2-Fluorophenyl)-1-(3-pyridyl)-1H-indole

### EXAMPLE 125

### 4-(2-Trifluoromethylphenyl)-1-(3-pyridyl)-1H-indole

### EXAMPLE 126

### 6-(2-Methoxyphenyl)-1-(3-pyridyl)-1H-indole

### EXAMPLE 127

### 4-(2-Acetylphenyl)-1-(3-pyridyl)-1H-indole

### Example 128

### 6-(3-Thienyl)-1-(3-pyridyl)-1H-indole

### EXAMPLE 129

### 1,4-bis(3-Pyridyl)-1H-indole

### EXAMPLE 130

### 1,6-bis(3-Pyridyl)-1H-indole

### EXAMPLE 131

### 1-(4-Methyl-3-pyridyl)₄-(4-trifluoromethylphenyl)-1H-indole

### EXAMPLE 132

### 6-(4-Methoxyphenyl)-1-(4-methyl-3-pyridyl)-1H-indole

### EXAMPLE 133

### 3-[1-(4-Methyl-3-pyridyl)-1H-indol-4-yl]benzonitrile

### EXAMPLE 134

### 1-(4-Methyl-3-pyridyl)-6-(3-pyrimidyl)-1H-indole

### EXAMPLE 135

### 1,6-bis(4-Methyl-3-pyridyl)-1H-indole

### EXAMPLE 136

### 6-(4-Fluorophenyl)-1-(4-methyl-3-pyridyl)-1H-indole

### EXAMPLE 137

### 4-(4-Cyanophenyl)-1-(4-methyl-3-pyridyl)-1H-indole

### EXAMPLE 138

### 6-(4-Chlorophenyl)-1-(4-methyl-3-pyridyl)-1H-indole

### EXAMPLE 139

### 1-(2-Fluorophenyl)₄-(3-pyridyl)-1H-indole

### EXAMPLE 140

### 1-(3-Cyanophenyl)-6-(3-pyridyl)-1H-indole

### EXAMPLE 141

### 1-(3-Fluoropheny)-4-(3-pyridyl)-1H-indole

### EXAMPLE 142

### 1-(2-Chlorophenyl)-6-(3-pyridyl)-1H-indole

### EXAMPLE 143

### 1-(2-Pyridyl)₄-(3-pyridyl)-1H-indole

### EXAMPLE 144

### 1-(2-Cyanophenyl)-6-(3-pyridyl)-1H-indole

### EXAMPLE 145

### 1-(3-Bromophenyl)-4-(3-pyridyl)-1H-indole

### EXAMPLE 146

### 1-(3-Chlorophenyl)-6-(3-pyridyl)-1H-indole

### EXAMPLE 147

### 4-(2-Pyridyl)-1-(3-pyridyl)-1H-indole

### EXAMPLE 148

### 1-(3-Pyridyl)-6-(4-pyridyl)-1H-indole

### EXAMPLE 149

### 1-Phenyl-4-(3-pyridyl)-1H-indole

### EXAMPLE 150

### 6-(4-Methyl-3-pyridyl)-1-(2-pyridyl)-1H-indole

### EXAMPLE 151

### 1-(3-Cyanophenyl)-4-(4-methyl-3-pyridyl)-1H-indole

### EXAMPLE 152

### 1-(2-Cyanophenyl)-6-(4-methyl-3-pyridyl)-1H-indole

### EXAMPLE 153

### 1-(4-Methyl-3-pyridyl)₄-(3-pyridyl)-1H-indole

### EXAMPLE 154

### 1-(4-Methyl-3-pyridyl)-6-(4-pyridyl)-1H-indole

### EXAMPLE 155

### 1,4-bis(4-Methyl-3-pyridyl)-1H-indole

### EXAMPLE 156

### 4-(3-Furyl)-1-(4-methyl-3-pyridyl)-1H-indole

### EXAMPLE 157

### 1-(4-Methyl-3-pyridyl)-6-(3-thienyl)-1H-indole

### EXAMPLE 158

### 1-(4-Methyl-3-pyridyl)-4-phenyl-1H-indole

### EXAMPLE 159

### 6-(2-Fluorophenyl)-1-(4-methyl-3-pyridyl)-1H-indole

### EXAMPLE 160

### 4-(2-Cyanophenyl)-1-(4-methyl-3-pyridyl)-1H-indole

### EXAMPLE 161

### 6-(2-Chlorophenyl)-1-(4-methyl-3-pyridyl)-1H-indole

### EXAMPLE 162

### 4-(3-Fluorophenyl)-1-(4-methyl-3-pyridyl)-1H-indole

### EXAMPLE 163

### 6-(3-Chlorophenyl)-1-(4-methyl-3-pyridyl)-1H-indole

### EXAMPLE 164

### 1-(4-Methyl-3-pyridyl)₄-(3-nitrophenyl)-1H-indole

The following intermediates can be prepared according to literature procedures:

5-Bromo4-fluoro-1H-indole (Laban, U. et al. Bio. Med. Chem. Lett. 2001, 11, 793-795).

5-Bromo-3-methyl-1H-indole (Le Borgne, M. et al. Bio. Med. Chem. Lett. 1999, 9, 333-336).

5-Bromo-3-cyano-1H-indole (Jiang, B. Bio. Med. Chem. 2000, 8, 363-371).

5-Bromo-7-chloro-1H-indole (Ezquerra, J. J. Org. Chem. 1996, 61, 5804-5812).

5-Bromo-2-methyl-1H-indole (Merour, J.-Y. et al. Syn. Comm. 1996,26,3267-3276).

5-Bromo-6-methoxy-1H-indole (Forbes, I. T. et al. PCT publication WO9602537, 1996).

5-Bromo-7-methyl-1H-indole (Ambekar, S. Y. et al. Monatsl. Chem. 1967, 98, 798-801).

5-Bromo-6-fluoro-1H-indole (Ackermann, J. et al. PCT publication WO0244149, 2002).

5-Bromo-3-isopropyl-1H-indole (Haning, H. et al. PCT publication WO0251805, 2002).

5-Bromo-7-methyl-1H-indazole (Dell'Erba, C. et al. Tetrahedron 1994, 50, 3529-3536).

Each of the above publications is hereby incorporated herein by reference.

Other 5-bromo-1H-indole and 5-bromo-1H-indazole intermediates can also be prepared by these or similar procedures using the appropriate starting materials.

The following compounds, as illustrative examples, can be prepared from the aforementioned intermediates by using the methods depicted in Scheme 1 and the appropriate reagents.

### EXAMPLE 165

### 4-Fluoro-1-(3-pyridyl)-5-(3-thienyl)-1H-indole

### EXAMPLE 166

### 4-Fluoro-5-phenyl-1-(3-pyridyl)-1H-indole

### EXAMPLE 167

### 4-Fluoro-1,5-bis(3-pyridyl)-1H-indole

### EXAMPLE 168

### 4-Fluoro-1,5-bis(4-methyl-3-pyridyl)-1H-indole

### EXAMPLE 169

### 3-[4-Fluoro-1-(4-methyl-3-pyridyl)-1H-indol-5-yl]benzonitrile

### EXAMPLE 170

### 4-Fluoro-5-(2-fluorophenyl)-1-(4-methyl-3-pyridyl)-1H-indole

### EXAMPLE 171

### 3-Methyl-5-phenyl-1-(3-pyridyl)-1H-indole

### EXAMPLE 172

### 3-Methyl-1,5-bis(4-methyl-3-pyridyl)-1H-indole

### EXAMPLE 173

### 3-[3-Methyl--(4-methyl-3-pyridyl)-1H-indol-5-yl]benzonitdile

### EXAMPLE 174

### 3-Cyano-5-phenyl-1-(3-pyridyl)-1H-indole

### EXAMPLE 175

### 3-Cyano-1,5-bis(4-methyl-3-pyridyl)-1H-indole

### EXAMPLE 176

### 3-[3-Cyano-1-(4-methyl-3-pyridyl)-1H-indol-5-yl]benzonitrle

### EXAMPLE 177

### 7-Chloro-1,5-bis(4-methyl-3-pyridyl)-1H-indole

### EXAMPLE 178

### 7-Chloro-5-(2-fluorophenyl)-1-(4-methyl-3-pyridyl)-1H-indole

### EXAMPLE 179

### 7-Chloro-1-(3-pyridyl)-5-(3-thienyl)-1H-indole

### EXAMPLE 180

### 7-Chloro-5-phenyl-1-(3-pyridyl)-1H-indole

### EXAMPLE 181

### 7-Chloro-1,5-bis(3-pyridyl)-1H-indole

### EXAMPLE 182

### 3-[7-Chloro-1-(4-methyl-3-pyridyl)-1H-indol-5-yl]benzonitrile

### EXAMPLE 183

### 2-Methyl-5-phenyl-1-(3-pyridyl)-1H-indole

### EXAMPLE 184

### 2-Methyl-1,5-bis(4-methyl-3-pyridyl)-1H-indole

### EXAMPLE 185

### 3-[2-Methyl-1-(4-methyl-3-pyridyl)-1H-indol-5-yl]benzonitrile

### EXAMPLE 186

### 6-Methoxy-1-(4-methyl-3-pyridyl)-5-(3-pyridyl)-1H-indole

### EXAMPLE 187

### 6-Methoxy-1-(4-methyl-3-pyridyl)-5-phenyl-1H-indole

### EXAMPLE 188

### 7-Methyl-1,5-bis(3-pyridyl)-1H-indole

### EXAMPLE 189

### 5-(3-Cyanophenyl)-7-methyl-1-(4-methyl-3-pyridyl)-1H-indole

### EXAMPLE 190

### 6-Pluoro-1,5-bis(4-methyl-3-pyridyl)-1H-indole

### EXAMPLE 191

### 6-Fluoro-5-(2-fluorophenyl)-1-(4-methyl-3-pyridyl)-1H-indole

### EXAMPLE 192

### 3-Isopropyl-1,5-bis(3-pyridyl)-1H-indole

### EXAMPLE 193

### 5-(3-Cyanophenyl)-3-isopropyl-1-(4-methyl-3-pyridyl)-1H-indole

### EXAMPLE 194

### 7-Methyl-1-(4-methyl-3-pyridyl)-5-(3-pyridyl)-1H-indazole

### EXAMPLE 195

### 5-(3-Cyanophenyl)-7-methyl-1-(4-methyl-3-pyridyl)-1H-indazole

The structures for the compounds of the invention, and data characterizing them, are shown in Tables 1-8 below.

TABLE 1

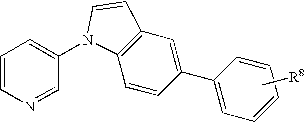

| Example | | TLC R_{f} (1:1 | LC/MS | HPLC* | Synthetic |
|---|---|---|---|---|---|
| # | R⁸ | EtOAc/Hex) | [M + 1]⁺ | Rt (min.) | Method |
| 1 | 4-Me | 0.40 | 285.4 | 3.24 | C |
| 2 | H | 0.28 | 271.4 | 3.07 | C |
| 3 | 4-Et | 0.40 | 299.4 | 3.43 | C |
| 4 | 4-t-Bu | 0.41 | 327.4 | 3.71 | C |
| 5 | 4-Cl | 0.40 | 305.4 | 3.37 | C |
| 6 | 4-F | 0.39 | 289.3 | 3.11 | C |
| 7 | 4-CF₃ | 0.39 | 339.4 | 3.44 | C |
| 8 | 4-OMe | 0.39 | 301.4 | 3.02 | C |
| 9 | 4-OEt | 0.39 | 315.3 | 3.22 | C |
| 10 | 4-CO₂Me | 0.34 | 329.4 | 3.07 | C |
| 11 | 4-CO₂H | — | 315.3 | 2.81 | C |
| 12 | 3-Me | 0.35 | 285.4 | 2.80 | C |
| 13 | 3-Cl | 0.45 | 305.4 | 3.91 | C |
| 14 | 3-F | — | 289.4 | 3.71 | C |
| 15 | 3-CF₃ | — | 339.4 | 3.97 | C |
| 16 | 3-OMe | — | 301.3 | 3.60 | C |
| 17 | 3-OEt | — | 315.3 | 3.79 | C |
| 18 | 3-NO₂ | — | 316.3 | 3.66 | C |
| 19 | 3-CN | 0.31 | | 3.33 | C |
| 20 | 2-Me | 0.37 | 285.4 | 3.67 | C |
| 21 | 2-Cl | 0.35 | 305.3 | 3.80 | C |
| 22 | 2-F | 0.41 | 289.4 | 3.67 | C |
| 23 | 2-CF₃ | 0.45 | 339.4 | 3.85 | C |
| 24 | 2-OMe | 0.39 | 301.3 | 3.59 | C |
| 25 | 2-COMe | 0.32 | 313.2 | 3.38 | C |

*HPLC Method: 10-90% 0.1% TFA in CH₃CN/0.1% TFA in water; 4 min. gradient; 6.5 min. total run time; C₁₈ ODS column (2 mm × 23 mm, 5 um).

TABLE 2

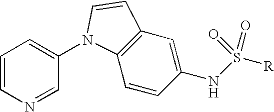

| Ex- | | | | | |
|---|---|---|---|---|---|
| ample | | TLC R_{f} (3:1 | LC/MS | HPLC* | Synthetic |
| # | R | EtOAc/Hex) | [M + 1]⁺ | Rt (min.) | Method |
| 30 | Me | 0.24 | 288.1 | 1.51 | F |
| 31 | Et | — | 302.1 | 1.95 | F |
| 32 | Ph | — | 350.1 | 2.39 | F |
| 33 | CH₂Ph | — | 364.2 | 2.54 | F |
| 34 | 4-F-Ph | — | 368.1 | 2.49 | F |
| 35 | 4-CN-Ph | — | 375.1 | 2.45 | F |
| 36 | 4-OMe-Ph | — | 380.1 | 2.44 | F |
| 37 | 4-NO₂-Ph | — | 395.1 | 2.59 | F |
| 38 | 4-CF₃-Ph | — | 418.1 | 2.83 | F |
| 39 | 4-COMe-Ph | — | 392.1 | 2.39 | F |
| 40 | 4-Me-Ph | — | 364.1 | 2.55 | F |
| 41 | 4-iPr-Ph | — | 392.2 | 2.87 | F |
| 42 | 4-tBu-Ph | — | 406.4 | 2.87 | F |
| 43 | 3-F-Ph | — | 368.2 | 2.56 | F |
| 44 | 2-F-Ph | — | 368.2 | 2.46 | F |
| 45 | 2-CF₃-Ph | — | 418.1 | 2.72 | F |
| 46 | 2-Me-Ph | — | 364.2 | 2.58 | F |
| 47 | 2-Cl-3-F-Ph | — | 402.1 | 2.67 | F |
| 48 | 3,4-F₂-Ph | — | 386.1 | 2.67 | F |
| 49 | 2,5-F₂-Ph | — | 386.1 | 2.56 | F |

*HPLC Method: 10-90% 0.1% TFA in CH₃CN/0.1% TFA in water; 4 min. gradient; 6.5 min. total run time; C₁₈ ODS column (2 mm × 23 mm, 5 um).

TABLE 3

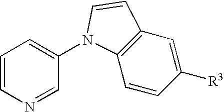

| Ex- | | | | | |
|---|---|---|---|---|---|
| am- | | | | | |
| ple | | | LC/MS | HPLC | Synthetic |
| # | R³ | TLC R_{f} | [M + 1]⁺ | Rt (min.) | Method |
| 50 | 1-piperidinyl | 0.2 (2:3 | 278.3 | 0.74 | G |
| | | EtOAc/Hex | | | |
| 51 | 1-pyrrolidinyl | 0.29 (1:2 | 264.2 | 0.83 | G |
| | | EtOAc/Hex | | | |
| 52 | NHPh | 0.29 (1:1 | 286.2 | 2.88 | H |
| | | EtOAc/Hex) | | | |
| 29 | 4-morpholinyl | — | 280.2 | 1.14 | D |
| 26 | 3-thienyl | 0.35 (1:1 | 277.3 | 3.38 | C |
| | | EtOAc/Flex) | | | |
| 27 | 3-furyl | 0.36 (1:1 | 261.1 | 3.15 | C |
| | | EtOAc/Hex) | | | |
| 28 | 3-pyridyl | 0.13 (1:1 | 272.3 | 1.36 | C |
| | | EtOAc/Hex) | | | |
| 62 | 2-pyridyl | 0.24 (1:1 | 272.3 | 0.75 | M |
| | | EtOAc/Hex) | | | |
| 63 | 4-pyridyl | 0.2 (EtOAc) | 272.4 | 1.64 | M |
| 61 | 2-Me-3-pyridyl | 0.27 (4:1 | 286.3 | 1.25 | M |
| | | EtOAc/Hex | | | |
| 64 | 2-Me-5-pyridyl | 0.21 (4:1 | 286.4 | 1.05 | M |
| | | EtOAc/Hex) | | | |
| 65 | 3-CN-5-pyridyl | 0.24 (3:2 | 297.3 | 2.43 | M |
| | | EtOAc/Hex) | | | |

TABLE 4

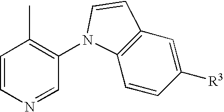

| Ex- | | | | | |
|---|---|---|---|---|---|
| ample | | | LCMS | HPLC | Synthetic |
| # | R³ | TLC R_{f} | [M + 1]⁺ | Rt (min.) | Method |
| 78 | Br | 0.31 (1:1 | 287.2 | 2.57 | P |
| | | EtOAc/Hex | | | |
| 79 | CN | 0.21 (1:1 | 234.3 | 2.68 | P |
| | | EtOAc/Hex) | | | |
| 80 | OCH₂Ph | 0.33 (1:1 | 315.2 | 3.46 | P |
| | | EtOAc/Hex | | | |
| 82 | 3-pyridyl | 0.26 (EtOAc) | 286.3 | 1.13 | Q |
| 83 | 4-pyridyl | 0.22 (EtOAc) | 286.4 | 1.05 | Q |
| 84 | 4-Me-3- | 0.3 (EtOAc) | 300.4 | 1.18 | Q |
| | pyridyl | | | | |
| 104 | 5- | — | 287.3 | 1.87 | R |
| | pyrimidyl | | | | |
| 85 | 3-furyl | 0.3 (1:1 | 275.5 | 2.53 | Q |
| | | EtOAc/Hex) | | | |
| 86 | 3-thienyl | 0.29 (1:1 | 291.3 | 2.70 | Q |
| | | EtOAc/Hex) | | | |
| 87 | Ph | 0.34 (1:1 | 285.3 | 2.93 | Q |
| | | EtOAc/Hex) | | | |
| 88 | 2-F-Ph | 0.28 (1:1 | 303.5 | 2.89 | Q |
| | | EtOAc/Hex | | | |
| 89 | 2-CN-Ph | 0.22 (1:1 | 310.3 | 2.72 | Q |
| | | EtOAc/Hex) | | | |
| 90 | 2-Cl-Ph | 0.31 (1:1 | 319.3 | 3.06 | Q |
| | | EtOAc/Hex | | | |
| 91 | 3-F-Ph | 0.28 (1:1 | 303.5 | 2.94 | Q |
| | | EtOAc/Hex | | | |
| 92 | 3-Cl-Ph | 0.32 (1:1 | 319.6 | 3.14 | Q |
| | | EtOAc/Hex) | | | |
| 103 | 3-CN-Ph | 0.25 (1:1 | 310.4 | 2.72 | Q |
| | | EtOAc/Hex) | | | |
| 93 | 3-NO₂-Ph | 0.25 (1:1 | 330.2 | 2.82 | Q |
| | | EtOAc/Hex | | | |
| 94 | 4-F-Ph | 0.26 (1:1 | 303.3 | 3.01 | Q |
| | | EtOAc/Hex | | | |
| 95 | 4-CN-Ph | 0.53 (EtOAc) | 310.4 | 2.83 | Q |
| 96 | 4-Cl-Ph | 0.3 (1:1 | 319.4 | 3.10 | Q |
| | | EtOAc/Hex) | | | |
| 97 | 4-CF₃-Ph | 0.31 (1:1 | 353.5 | 3.18 | Q |
| | | EtOAc/Hex) | | | |
| 98 | 4-OMe-Ph | 0.3 (1:1 | 315.4 | 2.78 | Q |
| | | EtOAc/Hex | | | |

TABLE 5

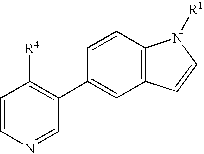

| Example | | | | LCMS | HPLC | Synthetic |
|---|---|---|---|---|---|---|
| # | R¹ | R⁴ | TLC R_{f} | [M + 1]⁺ | Rt (min.) | Method |
| 69 | Ph | H | 0.36 (1:1 | 271.3 | 2.31 | N |
| | | | EtOAc/Hex) | | | |
| 53 | 2-F-Ph | H | 0.29 (2:3 | 289.3 | 2.36 | J |
| | | | EtOAc/Hex) | | | |
| 56 | 2-Cl-Ph | H | 0.29 (2:3 | 305.3 | 2.46 | K |
| | | | EtOAc/Hex) | | | |
| 58 | 2-CN-Ph | H | 0.42 (3:2 | 296.3 | 2.17 | K |
| | | | EtOAc/Hex | | | |
| 54 | 3-CN-Ph | H | N/A | 296.4 | 2.16 | J |
| 55 | 3-F-Ph | H | 0.29 (2:3 | 289.4 | 2.31 | J |
| | | | EtOAc/Hex | | | |
| 59 | 3-Br-Ph | H | 0.29 (2:3 | 349.3 | 2.70 | K |
| | | | EtOAc/Hex | | | |
| 60 | 3-Cl-Ph | H | 0.29 (2:3 | 305.3 | 2.67 | K |
| | | | EtOAc/Hex) | | | |
| 57 | 2-pyridyl | H | 0.29 (1:1 | 272.4 | 2.02 | K |
| | | | EtOAc/Hex | | | |
| 99 | 2-Me-3- | H | 0.19 (EtOAc) | 286.4 | 0.91 | Q |
| | pyridyl | | | | | |
| 102 | 2-Me-5- | H | 0.09 (1:1 | 286.3 | 1.02 | Q |
| | pyridyl | | EtOAc/Hex | | | |
| 66 | 3-pyridyl | Me | 0.3 (EtOAc) | 286.3 | 1.54 | M |
| 70 | 2-pyridyl | Me | 0.26 (1:1 | 286.3 | 1.93 | N |
| | | | EtOAc/Hex) | | | |
| 71 | 3-CN-Ph | Me | 0.27 (1:1 | 310.4 | 2.14 | N |
| | | | EtOAc/Hex | | | |
| 72 | 2-CN-Ph | Me | 0.27 (3:2 | 310.4 | 2.06 | N |
| | | | EtOAc/Hex | | | |
| 77 | 3-CONH₂-Ph | Me | 0.23 (2% 2M | 328.4 | 1.81 | O |
| | | | NH₃/MeOH | | | |
| | | | in EtOAc) | | | |

TABLE 6

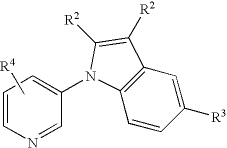

| Example | | | | | LCMS | HPLC | Synthetic |
|---|---|---|---|---|---|---|---|
| # | R⁴ | R² | R³ | TLC R_{f} | [M + 1]⁺ | Rt (min.) | Method |
| 100 | 2-Me | H | 4-F-Ph | 0.4 (EtOAc) | 303.3 | 2.76 | Q |
| 101 | 6-Me | H | 3-CN-Ph | 0.12 (1:2 | 310.4 | 2.65 | Q |
| | | | | EtOAc/Hex | | | |
| 68 | H | Me | 2-Cl-Ph | 0.23 (1:4 | 333.3 | 3.49 | N |
| | | | | EtOAc/Hex) | | | |
| 67 | H | Me | 3-pyridyl | 0.22 (2:1 | 300.4 | 1.89 | N |
| | | | | EtOAc/Hex) | | | |

TABLE 7

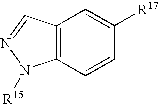

| Example | | | | LCMS | HPLC | Synthetic |
|---|---|---|---|---|---|---|
| # | R¹⁵ | R¹⁷ | TLC Rf | [M + 1]⁺ | Rt (min.) | Method |
| 73 | 3-pyridyl | 3-pyridyl | 0.23 (EtOAc) | 273.2 | 1.18 | N |
| 74 | 3-pyridyl | 4-F-Ph | 0.23 (1:2 | 290.3 | 2.85 | N |
| | | | EtOAc/Hex) | | | |
| 75 | 2-pyridyl | 4-Me-3- | 0.27 (1:1 | 287.3 | 1.94 | N |
| | | pyridyl | EtOAc/Hex) | | | |
| 81 | 4-Me-3- | 3-pyridyl | — | 287.2 | 0.92 | Q |
| | pyridyl | | | | | |

TABLE 8

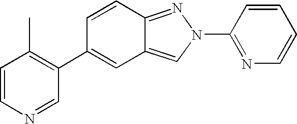

| Example | | LCMS | HPLC Rt | Synthetic |
|---|---|---|---|---|
| # | TLC R_{f} | [M + 1]⁺ | (min.) | Method |
| 76 | 0.27 (2:1 | 287.4 | 1.75 | N |
| | EtOAc/Hex) | | | |

Determination of the Activity of the Compounds of the Invention

C17,20-Lyase inhibitory activity of compounds can be determined using, e.g., the biochemical or the cellular assays set forth below. A person of skill in the art will recognize that variants of these assays can also be used.

The compounds of the invention can also be tested in animal models, e.g., animal models of prostate or breast cancer. Each of the compounds of the invention was subjected to a biochemical assay and a cellular assay for determining its C17,20 lyase inhibitory activity.

Human and murine C17,20-lyase biochemical assays:

Recombinant human C17,20-lyase (hLyase) was expressed in baculovirus-infected Sf9 cells and hLyase enriched microsomes were prepared from cultures as described (Barnes H. J.; Jenlins, C. M.; Waterman, M. R. Archives of Biochemistry and Biophysics 1994, 315(2), 489-494). Recombinant murine C17,20-lyase (mLyase) was prepared in a similar manner. hlyase and mLyase preparations were titrated using assay conditions to determine protein concentrations to be used for assays. Both mLyase and hLyase assays were run in an identical manner except that cytochrome b5 was omitted in the murine assay.

Test compound solutions (20 mM in DMSO) were diluted 1:4 with DMSO and put into the top well of a 96-well mother plate. These solutions were then diluted serially in six steps (1:4 each step) with DMSO to obtain 800 μM to 51.2 nM concentrations on a mother plate (columns 3-12) for subsequent use in the assay. These compound solutions were further diluted twenty-fold in water to obtain a daughter plate containing compound concentrations ranging from 40 μM to 2.56 nM in 5% DMSO. The first 2 columns (of wells) on each 96-well mother plate were used for the DHEA (dehydroepiandrosterone) standard curve. DHEA standards were serially diluted (in half-logs) in DMSO to obtain 400 μM to 120 nM standards, then diluted (1:19) in water to obtain 20 μM to 6 nM solutions in 5% DMSO on the daughter plate. These 5% DMSO solutions (5 μL each) from the daughter plate were transferred to the SPA assay plate prior to adding the reaction mixture.

To prepare the reaction mixture, clear-bottomed opaque 96-well assay plates were loaded with 50 μL of assay buffer (50 mM Na₃PO₄, pH 7.5), 5 mL of the diluted compounds (or standards), and 30 mL of substrate solutions (7 mM NADPH, 3.35 μM 17-OH-pregnenolone, 3.35 μg/mL human cytochrome b₅ in 50 mM Na₃PO₄). Reactions were initiated with the addition of hLyase or mLyase in assay buffer (10 μL). Enzymatic reactions were incubated at room temperature for 2 hours with gentle agitation. Reactions were terminated with the addition of 5 μL of 1 mM (50 μM final concentration) YM116, a potent C17,20-lyase inhibitor.

The concentration of DHEA generated by hLyase (or mLyase) was determined by radioimmunoassay (RIA). RIA utilized a ³H-DHEA (0.08 μCi) tracer in 50 μL of scintillation proximity assay (SPA) buffer (100 mM Tris-HCl, pH 7.5, 50 mM NaCl, 0.5% BSA, 0.2% Tween 20) which was added to each well. DHEA antiserum from rabbit (50 μL) with anti-rabbit SPA beads in SPA buffer was added to all wells. Mixtures were allowed to equilibrate with gentle agitation for 1 hour followed by overnight equilibration with no agitation. H-DHEA bound to the SPA beads was determined by scintillation counting with a Wallac microbeta counter. The concentration of DHEA generated was calculated from raw data (CPM) and the standard curve. The concentration of DHEA formed in the presence of test compounds was expressed as a percent inhibition compared to the DHEA concentration in the absence of test compounds: [1—(nM DHEA formed in the presence of test compound/nM DHEA formed in the absence of test compounds)]×100. Determination of IC₅₀ for each compound was performed using the Analyze 5 program.

Human C17,20-lyase cellular assay:

Human HEK 293-lyase stable transfectant cells were seeded in a 96-well plate at 10,000 cells/well/100 μL in DMEM plus 10% FBS (supplemented with 1% glutamine, 0.8 mg/mL G418) and allowed to attach overnight. On the following day, the media was removed from the cell plate and replaced with 100 μL RPMI without phenol red. Test compounds (columns 3-12), DMSO vehicle (column 2), or DHEA standards (column 1) of 5 mL each were added to the cell plate and incubated for 10 min. at room temperature. The final concentrations of DHEA standards were 750, 250, 83.3, 27.7, 9.2, 3, 1, and 0.3 nM. The reaction was initiated with 10 μL of 5 μM 17-OH-pregnenolone being added to all the wells of the cell plate, then incubated for 1 hour at 37° C. Following the incubation, 90 μL of media (containing DHEA product) was removed from the cell plate and transferred to the SPA assay plate. The subsequent SPA procedure for the detection of DHEA product was performed in the same manner as described for the enzyme assay (see above). The mother plate of test compounds was also prepared in the same manner as the enzyme assay. However, the highest concentration of compounds on the daughter plate was 200 μM rather than 40 μM, such that the highest dose of compound tested was 10 μM in final concentration (cellular assay) rather than 2 μM (biochemical assay).

Reagents (including catalog #) for the SPA assay were obtained from the following sources: ³H-DHEA: NEN (NET814), Anti-DHEA: Endocrine Sciences (D7-421), Anti-Rabbit SPA Beads: Amersham (RPNQ 0016), 17-OH-pregnenolone: Steraloids (Q4710), NADPH: Sigma (N1630), Cytochrome b5: Panvera (P2252), DHEA (500 μM stock in 100% EtOH), BSA: Sigma (A9647).

A test compound was considered to be active if the IC₅₀ in the human C17,20 biochemical assay or the human C17,20 cellular assay was less than 10 μM.

All the compounds tested have IC₅₀ in the human C17,20 biochemical assay or the human C17,20 cellular assay of less than 10 μM.

Other embodiments of the invention will be apparent to the skilled in the art from a consideration of this specification or practice of the invention disclosed herein. It is intended that the specification and examples be considered as exemplary only, with the true scope and spirit of the invention being indicated by the following claims.

We claim:

1. A compound of the formula 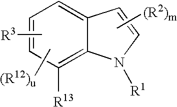
wherein
R¹ represents 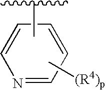
wherein R⁴ represents C₁₋₄ alkyl; and
p is 0, 1, or 2; 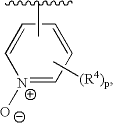
provided that R³ is other than a pyridyl or an N-oxide-containing group; or 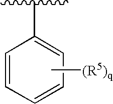
wherein
R⁵ represents
CN,
halogen,
CHO, or
C(O)N(R⁶)₂ wherein R⁶ represents H or C₁₋₄ allyl; and
q is 0, 1, or 2;
R² represents C₁₋₄ alkyl; and
m is 0, 1, or 2; and
R³ represents 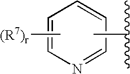
wherein
R⁷ is C₁₋₄ alkyl or CN; and
r is 0, 1, or 2; 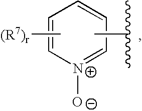
provided that R¹ is other than a pyridyl or an N-oxide-containing group; 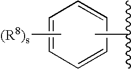
wherein
R⁸ represents
CN,
halogen,
C₁₋₄ alkyl,
C₁₋₄ alkoxy,
NO₂,
CF₃,
C₁₋₄ acyl,
CO₂R⁹ wherein R⁹ is H or C₁₋₄ alkyl, or 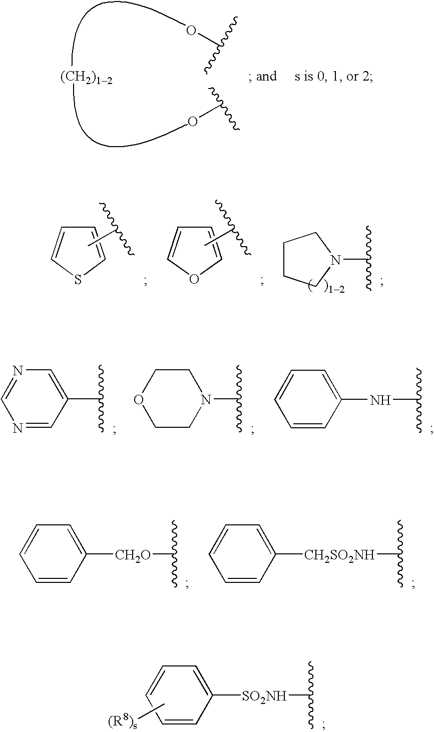
C₁₋₄ alkyl-SO₂NH—;
CN;
N(R¹⁰)₂, wherein R¹⁰ is C₁₋₄ alkyl; or 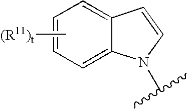
wherein
R¹¹ is halogen; and
t is 0, 1, or 2;
R¹² represents C₁₋₄ alkyl, C₁₋₄ alkoxy, halogen, or CN provided that R³ is other than cyano; and u is 0, 1, or 2;
R¹³ represents H or R¹²; and
one of R¹ and R³ is a 3-pyridyl or 3-pyridyl-N-oxide group which is unsubstituted at the 2- and 6-positions;
or a pharmaceutically acceptable salt thereof.

2. A compound according to claim 1
wherein
R¹ represents 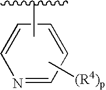
wherein R⁴ represents C₁₋₄ alkyl; and
p is 0, 1, or 2; 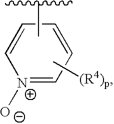
provided that R³ is other than a pyridyl or an N-oxide-containing group; or 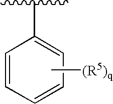
wherein
R⁵ represents
CN,
halogen,
CHO, or
C(O)N(R⁶)₂ wherein R⁶ represents H or C₁₋₄ alkyl; and
q is 0, 1, or 2;
R² represents C₁₋₄ alkyl; and
m is 0, 1, or 2; and
R³ represents 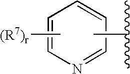
wherein
R⁷ is C₁₋₄ alkyl or CN; and
r is 0, 1, or 2; 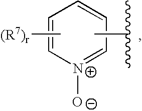
provided that R¹ is other than a pyridyl or an N-oxide-containing group; 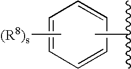
wherein
R⁸ represents
CN,
halogen,
C₁₋₄ alkyl,
C₁₋₄ alkoxy,
NO₂,
CF₃,
C₁₋₄ acyl,
CO₂R⁹ wherein R⁹ is H or C₁₋₄ alkyl, or

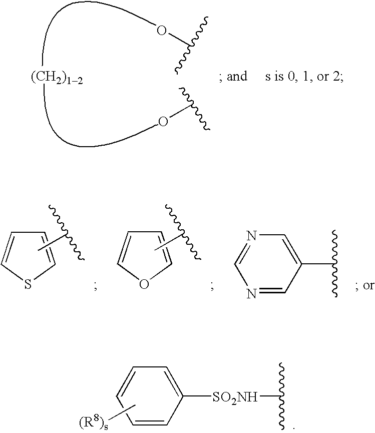

3. A compound according to claim 1
wherein
R¹ represents 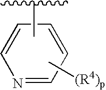
wherein R⁴ represents C₁₋₄ alkyl; and
p is 0, 1, or 2; 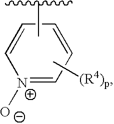
provided that R³ is other than a pyridyl or an N-oxide-containing group; or 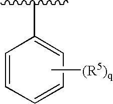
wherein
R⁵ represents
CN,
halogen,
CHO, or
C(O)N(R⁶)₂ wherein R⁶ represents H or C₁₋₄ alkyl; and
q is 0, 1, or 2;
R² represents C₁₋₄ alkyl; and
m is 0, 1, or 2; and
R³ represents 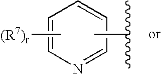
wherein
R⁷ is C₁₋₄ alkyl or CN; and
r is 0, 1, or 2; 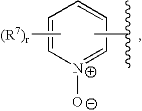
provided that R¹ is other than a pyridyl or an N-oxide-containing group; 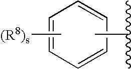
wherein
R⁸ represents
CN,
halogen,
C₁₋₄ alkyl,
C₁₋₄ alkoxy,
NO₂,
CF₃,
C₁₋₄ acyl,
CO₂R⁹ wherein R⁹ is H or C₁₋₄ alkyl, or

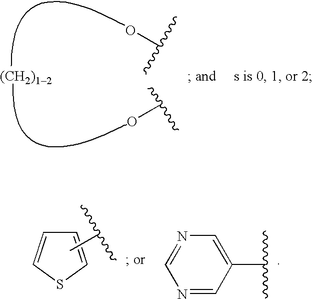

4. A compound according to claim 1
wherein
R¹ represents 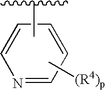
wherein R⁴ represents C₁₋₄ alkyl; and
p is 0, 1, or 2; or 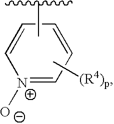
provided that R³ is other than a pyridyl or an N-oxide-containing group;
R² represents C₁₋₄ alkyl; and
m is 0, 1, or 2; and
R³ represents 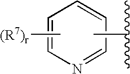
wherein
R⁷ is C₁₋₄ alkyl or CN; and
r is 0, 1, or 2;

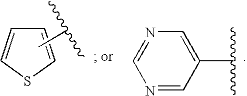

5. A pharmaceutical composition comprising a compound of claim 1 and a pharmaceutically acceptable carrier.

6. A method of inhibiting a lyase enzyme, comprising contacting said lyase enzyme with a compound of claim 1.

7. A method of inhibiting a 17α-hydroxylase-C17,20-lyase, comprising contacting a 17α-hydroxylase-C17,20-lyase with a compound of claim 1.

8. A method for treating a subject having a cancer associated with a 17α-hydroxylase-C17,20-lyase, comprising administering to the subject a therapeutically effective amount of a compound of claim 1.

9. A method for treating prostate cancer in a subject, comprising administering to said subject a therapeutically effective amount of a compound of claim 1, such that the prostate cancer in the subject is treated.

10. A method for treating breast cancer in a subject, comprising administering to said subject a therapeutically effective amount of a compound of claim 1, such that the breast cancer in the subject is treated.

11. The method of any one of claims 8-10, wherein said subject is a primate, equine, canine or feline.

12. The method of any one of claims 8-10, wherein said subject is a human.

13. A compound of the formula 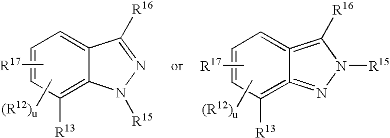
wherein
R¹² represents C₁₋₄ alkyl, C₁₋₄ alkoxy, halogen, or CN; and u is 0, 1, or 2;
R¹³ represents H or R¹²;
R¹⁵ represents 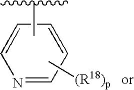
wherein R¹⁸ represents C₁₋₄ alkyl; and
p is 0, 1, or 2; or 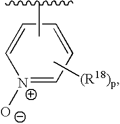
provided that R¹⁷ is other than a pyridyl or an N-oxide-containing group;
R¹⁶ represents H or C₁₋₄ alkyl; and
R¹⁷ represents 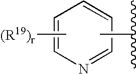
wherein
R¹⁹ is C₁₋₄ alkyl; and
r is 0, 1, or 2; or 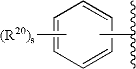
wherein
R²⁰ represents
halogen;
C₁₋₄ alkyl,
C₁₋₄ alkoxy,
NO₂,
CF₃, or
CO₂R²¹ wherein R²¹ is H or C₁₋₄ alkyl; and
s is 0, 1, or 2; and
one of R¹⁵ and R¹⁷ is a 3-pyridyl or 3-pyridyl-N-oxide group which is unsubstituted at the 2- and 6-positions;
or a pharmaceutically acceptable salt thereof.

14. A compound according to claim 13
wherein
R¹⁵ represents 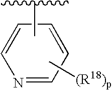
wherein R¹⁸ represents C₁₋₄ alkyl; and
p is 0, 1, or 2; or 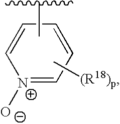
provided that R¹⁷ is other than a pyridyl or an N-oxide-containing group;
R¹⁶ represents H; and
R¹⁷ represents 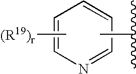
wherein
R¹⁹ is C₁₋₄ alkyl, and
r is 0, 1, or 2; or 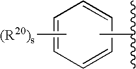
wherein
R²⁰ represents
halogen;
C₁₋₄ alkyl,
C₁₋₄ alkoxy,
NO₂,
CF₃, or
CO₂R²¹ wherein R²¹ is H or C₁₋₄ alkyl; and
s is 0, 1, or 2.

15. A compound according to claim 13
wherein
R¹⁵ represents 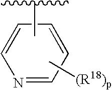
wherein R¹⁸ represents C₁₋₄ alkyl; and
p is 0, 1, or 2;
R¹⁶ represents H; and
R¹⁷ represents 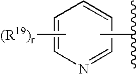
wherein
R¹⁹ is C₁₋₄ alkyl; and
r is 0, 1, or 2.

16. A pharmaceutical composition comprising a compound of claim 13 and a pharmaceutically acceptable carrier.

17. A method of inhibiting a lyase enzyme, comprising contacting said lyase enzyme with a compound of claim 13.

18. A method of inhibiting a 17α-hydroxylase-C17,20 lyase, comprising contacting a 17α-hydroxylase-C17,20 lyase with a compound of claim 13.

19. A method for treating a subject having a cancer associated with a 17α-hydroxylase-C17,20 lyase, comprising administering to the subject a therapeutically effective amount of a compound of claim 13.

20. A method for treating prostate cancer in a subject, comprising administering to said subject a therapeutically effective amount of a compound of claim 13, such that the prostate cancer in the subject is treated.

21. A method for treating breast cancer in a subject, comprising administering to said subject a therapeutically effective amount of a compound of claim 13, such that the breast cancer in the subject is treated.

22. The method of any one of claims 19-21, wherein said subject is a primate, equine, canine or feline.

23. The method of any one of claims 19-21, wherein said subject is a human.
